(19) United States
(12) Patent Application Publication
(10) Pub. No.: US 2012/0091445 A1
(43) Pub. Date: Apr. 19, 2012

(54) COMPOUND FOR ORGANIC OPTOELECTRONIC DEVICE, ORGANIC LIGHT EMITTING DIODE INCLUDING THE SAME, AND DISPLAY DEVICE INCLUDING THE ORGANIC LIGHT EMITTING DIODE

(76) Inventors: Sung-Hyun JUNG, Uiwang-si (KR); Hyung-Sun Kim, Uiwang-si (KR); Ho-Jae Lee, Uiwang-si (KR); Eun-Sun Yu, Uiwang-si (KR); Mi-Young Chae, Uiwang-si (KR); Young-Hoon Kim, Uiwang-si (KR); Ja-Hyun Kim, Boryeong-city (KR)

(21) Appl. No.: 13/335,991

(22) Filed: Dec. 23, 2011

Related U.S. Application Data
(63) Continuation of application No. PCT/KR2010/004157, filed on Jun. 25, 2010.

(30) Foreign Application Priority Data
Jun. 25, 2009 (KR) 10-2009-0057236

(51) Int. Cl. H01L 51/50; C07D 403/12; C07D 403/10; C07D 403/14; C07D 401/14

(52) U.S. Cl. 257/40; 544/209; 546/276.7; 544/294; 548/418; 257/E51.019

(57) ABSTRACT

A compound for an organic optoelectronic device, an organic light emitting diode, and a display device, the compound including substituents represented by the following Chemical Formulae 1 and 2:

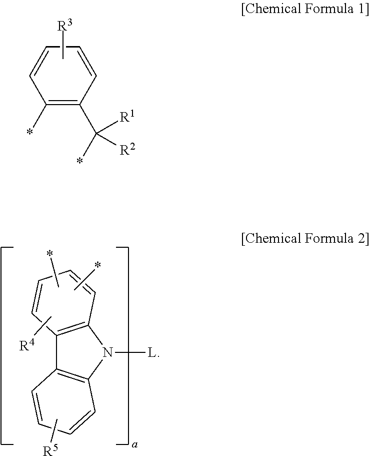

### CROSS-REFERENCE TO RELATED APPLICATION

This application is a continuation of pending International Application No. PCT/KR2010/004157, entitled “Compound for Organic Photoelectric Device and Organic Photoelectric Device Including the Same,” which was filed on Jun. 25, 2010, the entire contents of which are hereby incorporated by reference.

### BACKGROUND

1. Field

Embodiments relate to a compound for an organic optoelectronic device, an organic light emitting diode including the same, and a display device including the organic light emitting diode.

2. Description of the Related Art

An organic optoelectronic device is, in a broad sense, a device for transforming photo-energy to electrical energy or, conversely, a device for transforming electrical energy to photo-energy. As examples, organic optoelectronic devices may include an organic light emitting diode (OLED), a solar cell, a transistor, and the like. An organic light emitting diode has recently drawn attention due to the increase in demand for flat panel displays.

When current is applied to an organic light emitting diode, holes are injected from an anode and electrons are injected from a cathode. Then, injected holes and electrons move to a respective hole transport layer (HTL) and electron transport layer (ETL) and recombine to form a light emitting exciton in an emission layer. The light emitting excitons generate light while shifting to a ground state. The light emission material may be classified as a fluorescent material (using singlet excitons) and a phosphorescent material (using triplet excitons) according to light emitting mechanism. The fluorescent and phosphorescent materials may be used for a light emitting source of an organic light emitting diode.

When electrons are transported from the ground state to the exited state, a singlet exciton may undergo non-light emitting transition to a triplet exciton through intersystem crossing, and the triplet exciton may be transited to the ground state to emit light. Such light emission is referred to as phosphorescent emission. When the triplet exciton is transited, it may not directly transit to the ground state. Therefore, it may be transited to the ground state after the electron spin is flipped. Accordingly, a half-life (light emitting time, lifetime) of phosphorescent emission is longer than that of fluorescent emission.

When holes and electrons are recombined to produce a light emitting exciton, three times as many triplet light emitting excitons may be produced, compared to the amount of the singlet light emitting excitons. A fluorescent material has 25% of the singlet-exited state and a limit in luminous efficiency. On the other hand, a phosphorescent material may utilize 75% of the triplet exited state and 25% of the singlet exited state, so it may theoretically reach 100% of the internal quantum efficiency. Accordingly, the phosphorescent light emitting material may have advantages of accomplishing around four times greater luminous efficiency than the fluorescent light emitting material.

### SUMMARY

Embodiments are directed to a compound for an organic optoelectronic device, an organic light emitting diode including the same, and a display device including the organic light emitting diode.

The embodiments may be realized by providing a compound for an organic optoelectronic device including substituents represented by the following Chemical Formulae 1 and 2:

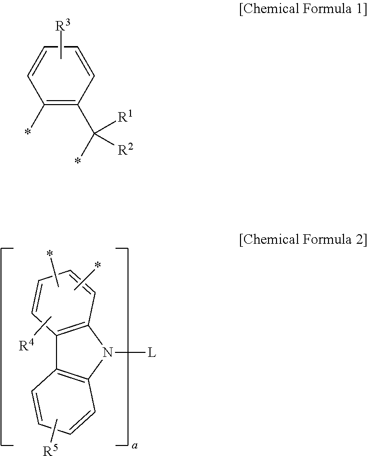

wherein, in Chemical Formulae 1 and 2 L is a divalent to heptavalent linking group, the divalent to heptavalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof, R¹ to R⁵ are each independently hydrogen, a substituted or unsubstituted carbazolyl group, a substituted or unsubstituted C1 to C30 alkyl group, a substituted or unsubstituted C6 to C30 aryl group, a substituted or unsubstituted C2 to C30 heteroaryl group, a substituted or unsubstituted C6 to C30 arylamine group, or a combination thereof, and a is an integer of 2 to 5.

R¹ and R² in Chemical Formula 1 may each independently be a carbazolyl group, a C1 to C30 alkyl group, a C6 to C30 aryl group, a C2 to C30 heteroaryl group, or a combination thereof.

R³ to R⁵ in Chemical Formulae 1 and 2 may each independently be hydrogen, a C1 to C30 alkyl group, a C6 to C30 aryl group, or a combination thereof.

The substituent represented by Chemical Formula 1 may be represented by the following Chemical Formula 1a:

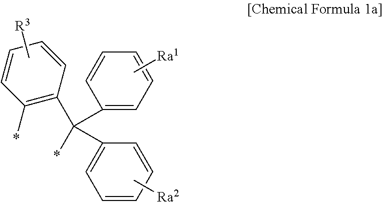

wherein in Chemical Formula 1a Ra¹ and Ra² may each independently be hydrogen or a C1 to C10 alkyl group, and R³ may be hydrogen, a carbazolyl group, a C1 to C30 alkyl group, a C6 to C30 aryl group, a C2 to C30 heteroaryl group, a C6 to C30 arylamine group, or a combination thereof.

The substituent represented by Chemical Formula 2 may be represented by one of the following Chemical Formulae 2a to 2c:

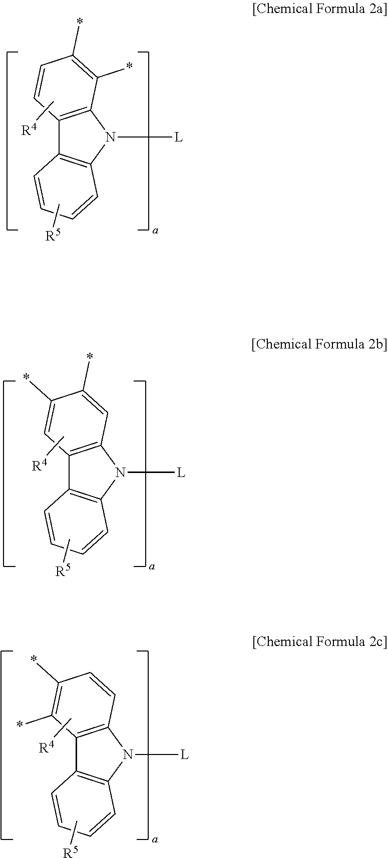

wherein, in Chemical Formulae 2a to 2c L may be a divalent to heptavalent linking group, the divalent to heptavalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof, R⁴ and R⁵ may each independently be hydrogen, a carbazolyl group, a C1 to C30 alkyl group, a C6 to C30 aryl group, a C2 to C30 heteroaryl group, a C6 to C30 arylamine group, or a combination thereof, and a may be an integer of 2 to 5.

The divalent to heptavalent linking group of L of Chemical Formula 2 may be derived from a compound represented by one of the following Chemical Formulae 2d to 2j, or a combination thereof:

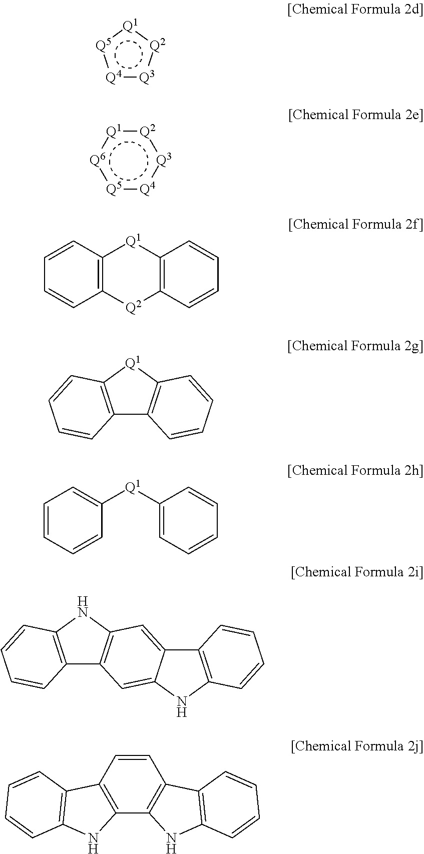

wherein, in Chemical Formulae 2d to 2j Q¹ to Q⁶ may each independently be a substituted or unsubstituted N atom, a substituted or unsubstituted P atom, a substituted or unsubstituted S atom, a substituted or unsubstituted O atom, a substituted or unsubstituted C atom, or a combination thereof, in which substituted refers to one substituted with an oxide group, a cyano group, a halogen group, a C1 to C30 alkyl group, a C6 to C30 aryl group, a C2 to C30 heteroaryl group, or a combination thereof.

In Chemical Formula 2, a may be 2 or 3.

The divalent to heptavalent linking group of L of the above Chemical Formula 2 may be represented by one of the following Formulae L-1 to L-117:

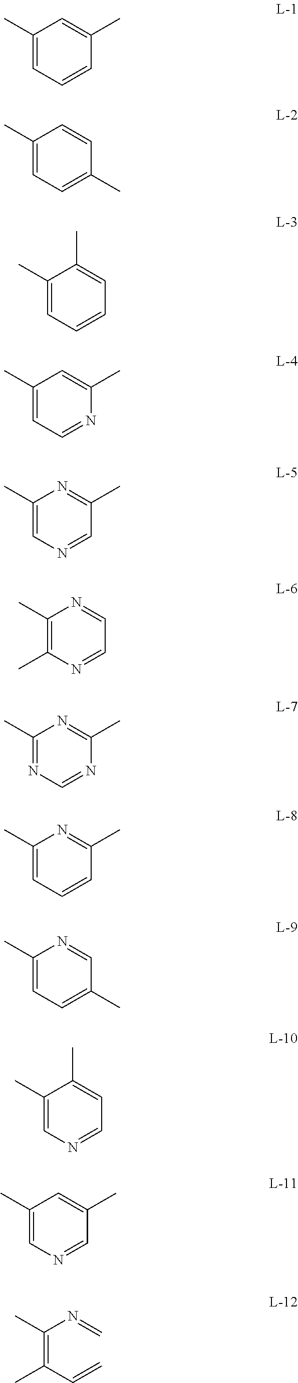

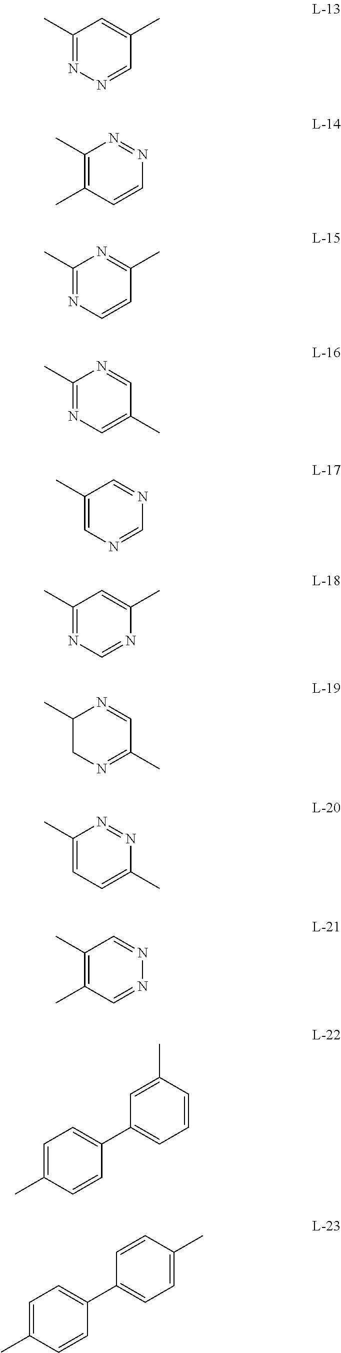

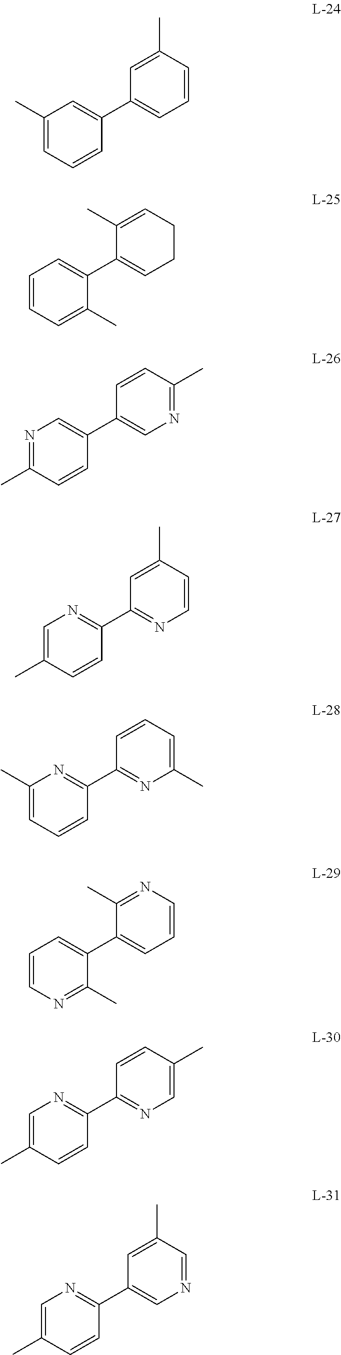

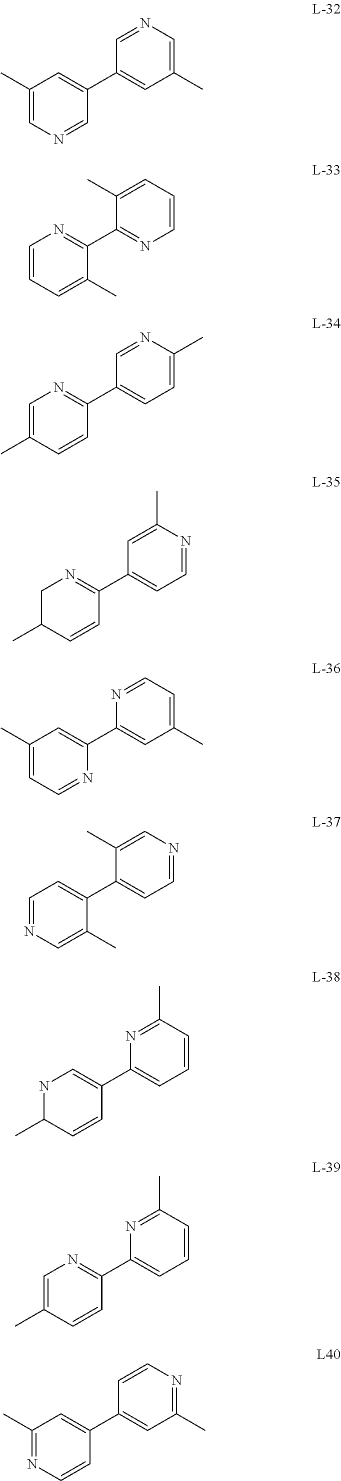

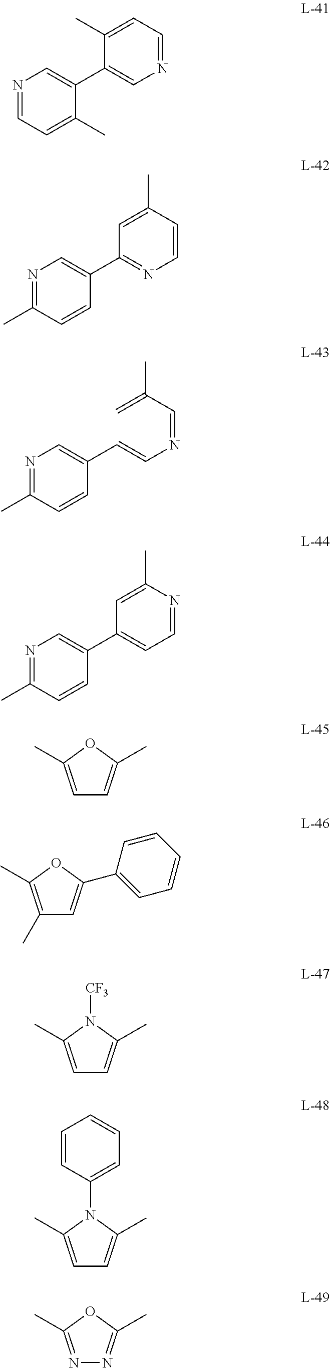

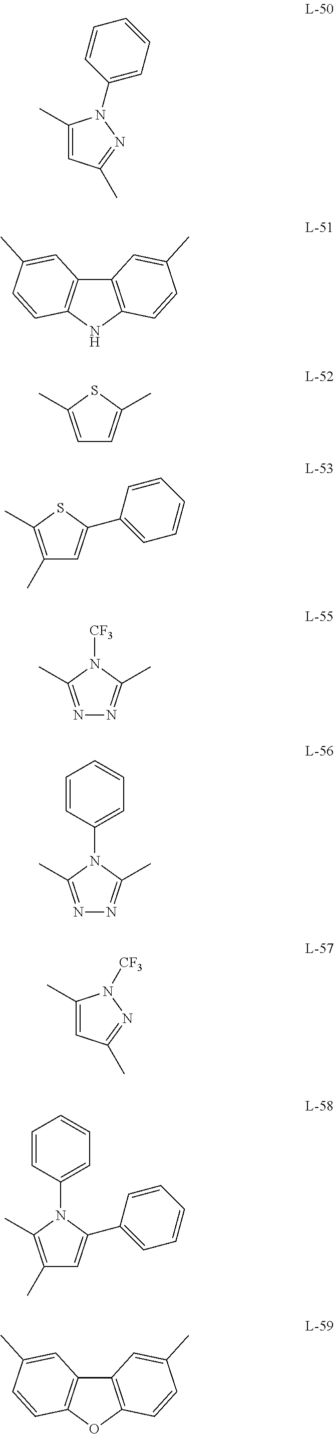

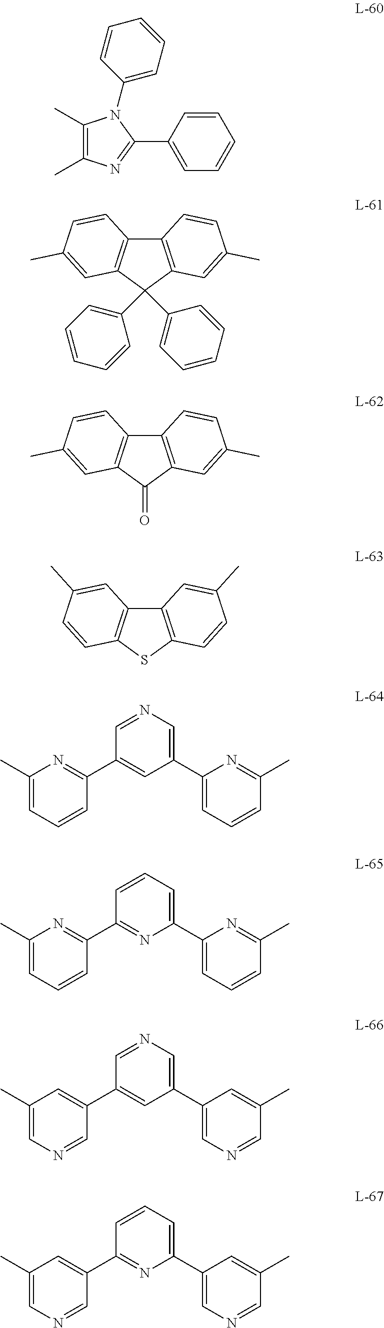

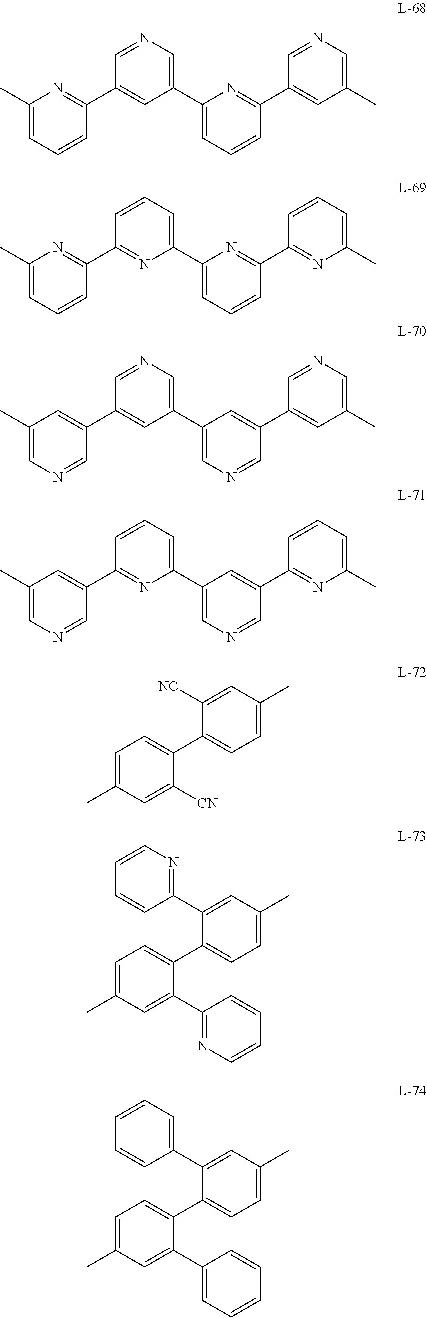

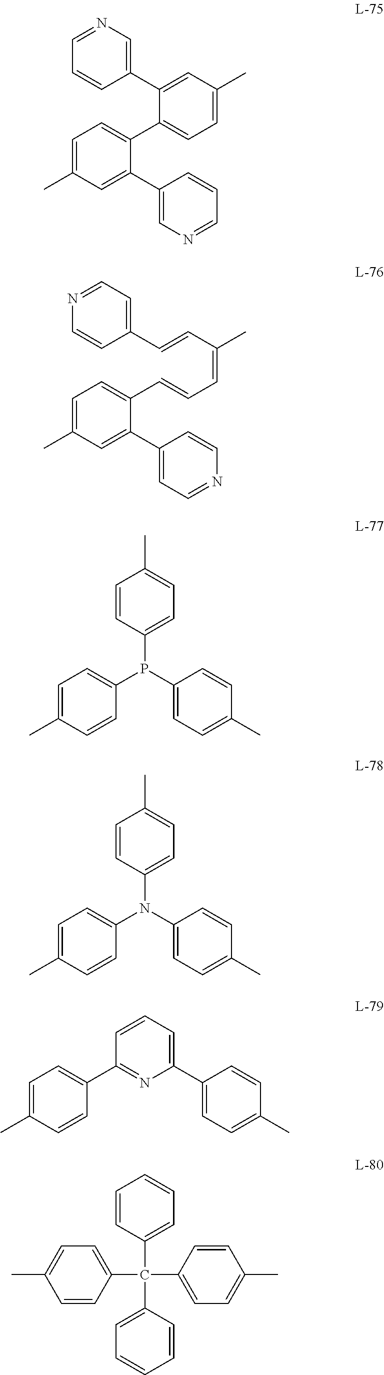

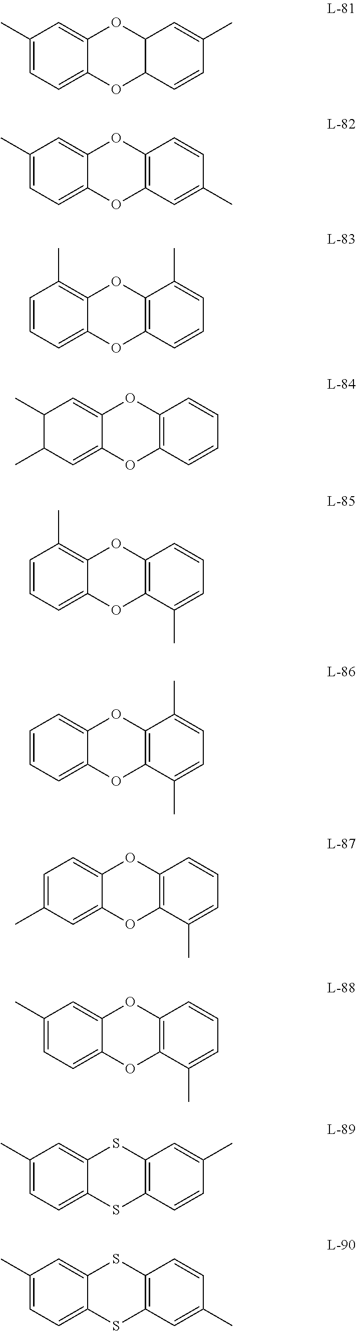

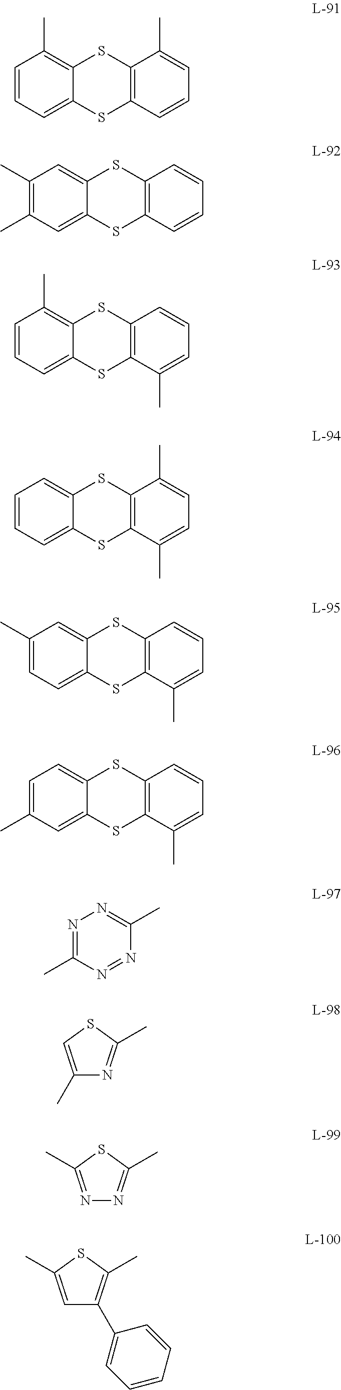

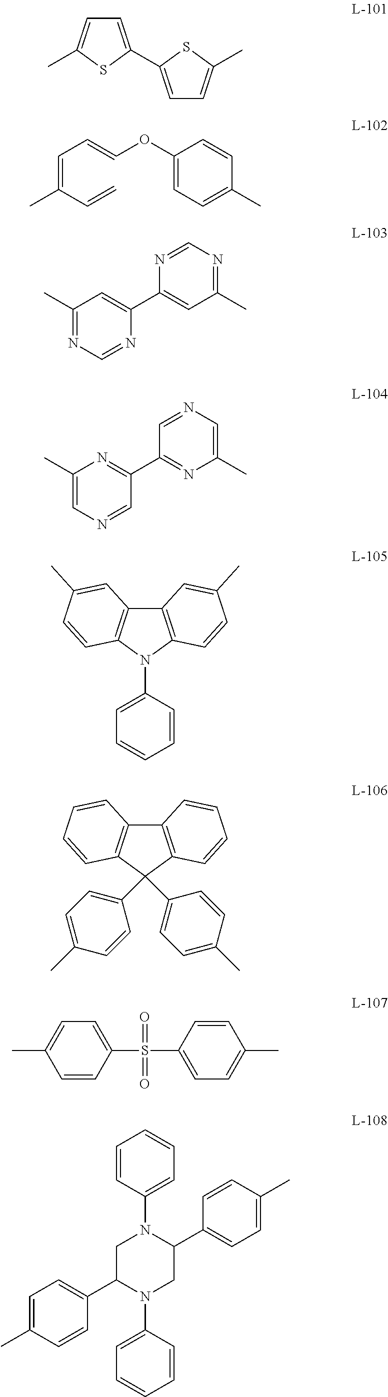

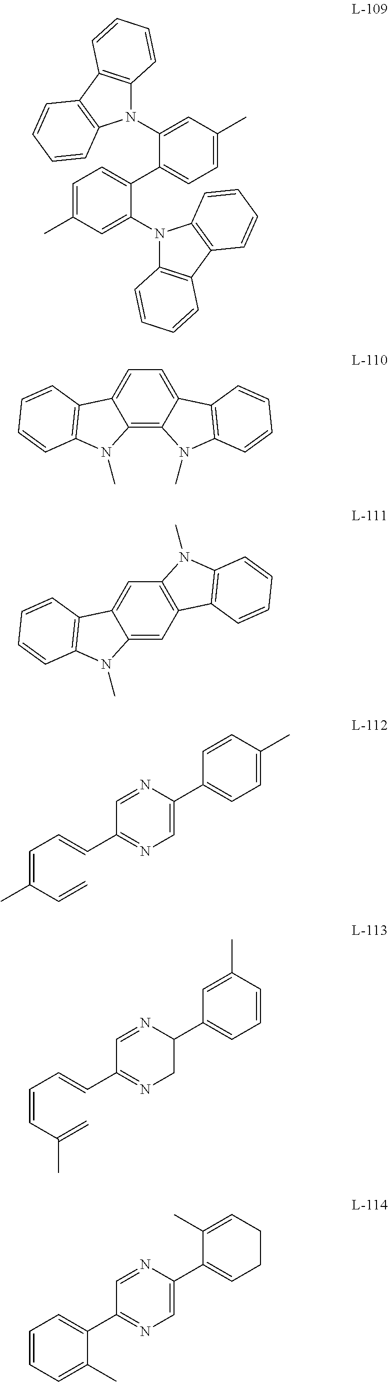

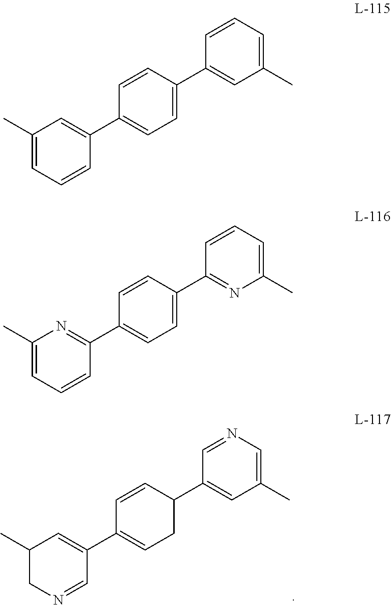

The compound for an organic optoelectronic device including the substituents represented by Chemical Formulae 1 and 2 may be represented by one of the following Chemical Formulae 3 to 8:

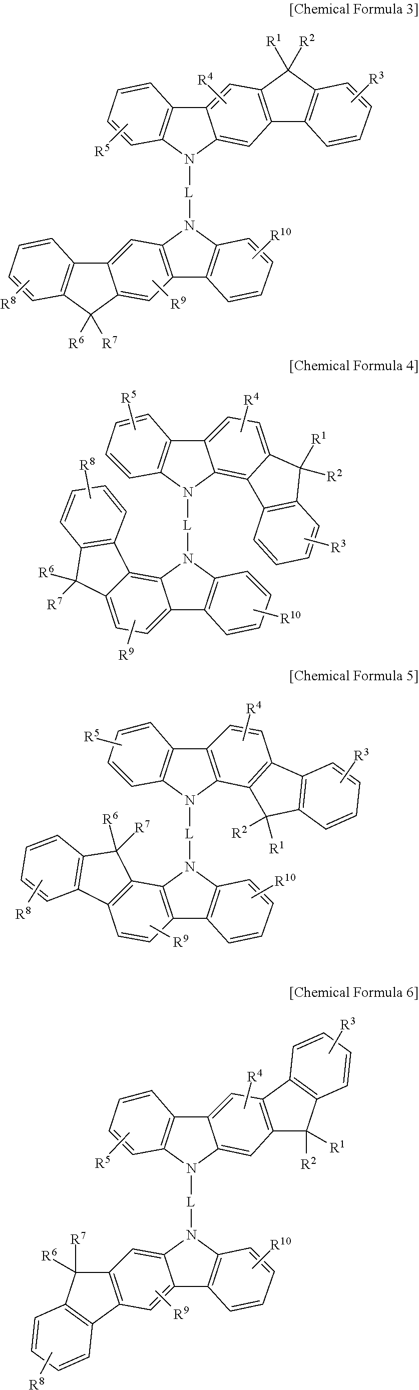

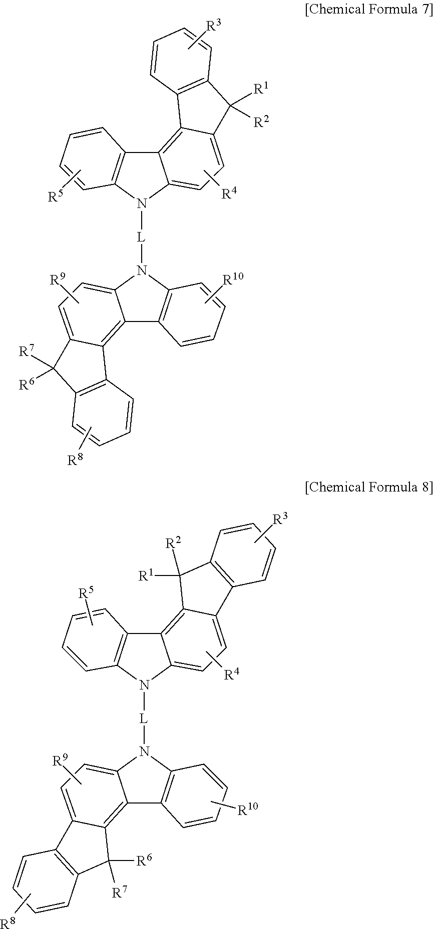

wherein, in Chemical Formulae 3 to 8 L may be a divalent linking group, the divalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof, and R¹ to R¹⁰ may each independently be hydrogen, a carbazolyl group, a C1 to C30 alkyl group, a C6 to C30 aryl group, a C2 to C30 heteroaryl group, a C6 to C30 arylamine group, or a combination thereof.

The compound for an organic optoelectronic device including the substituents represented by Chemical Formulae 1 and 2 may be represented by one of the following Chemical Formulae 9 to 33:

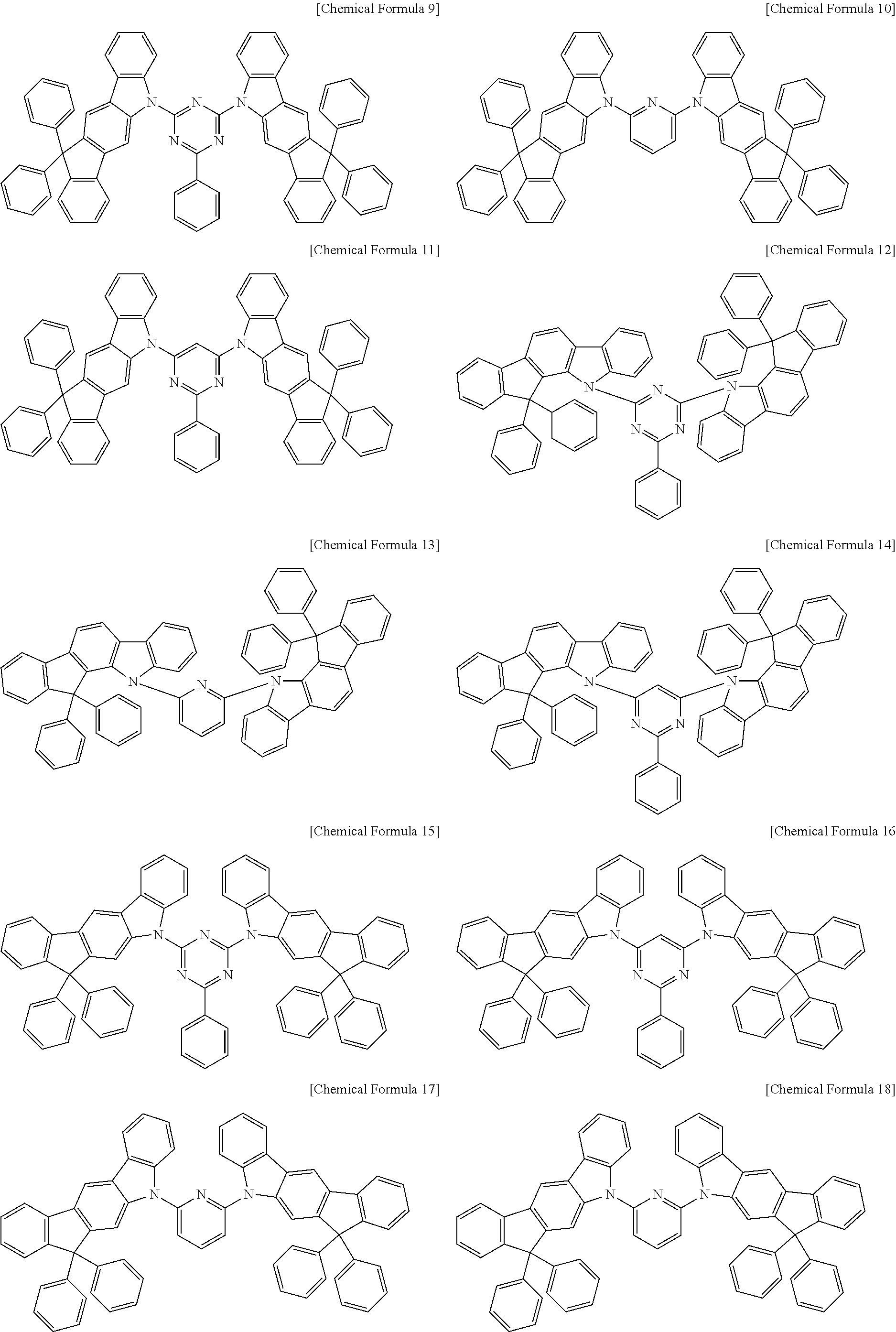

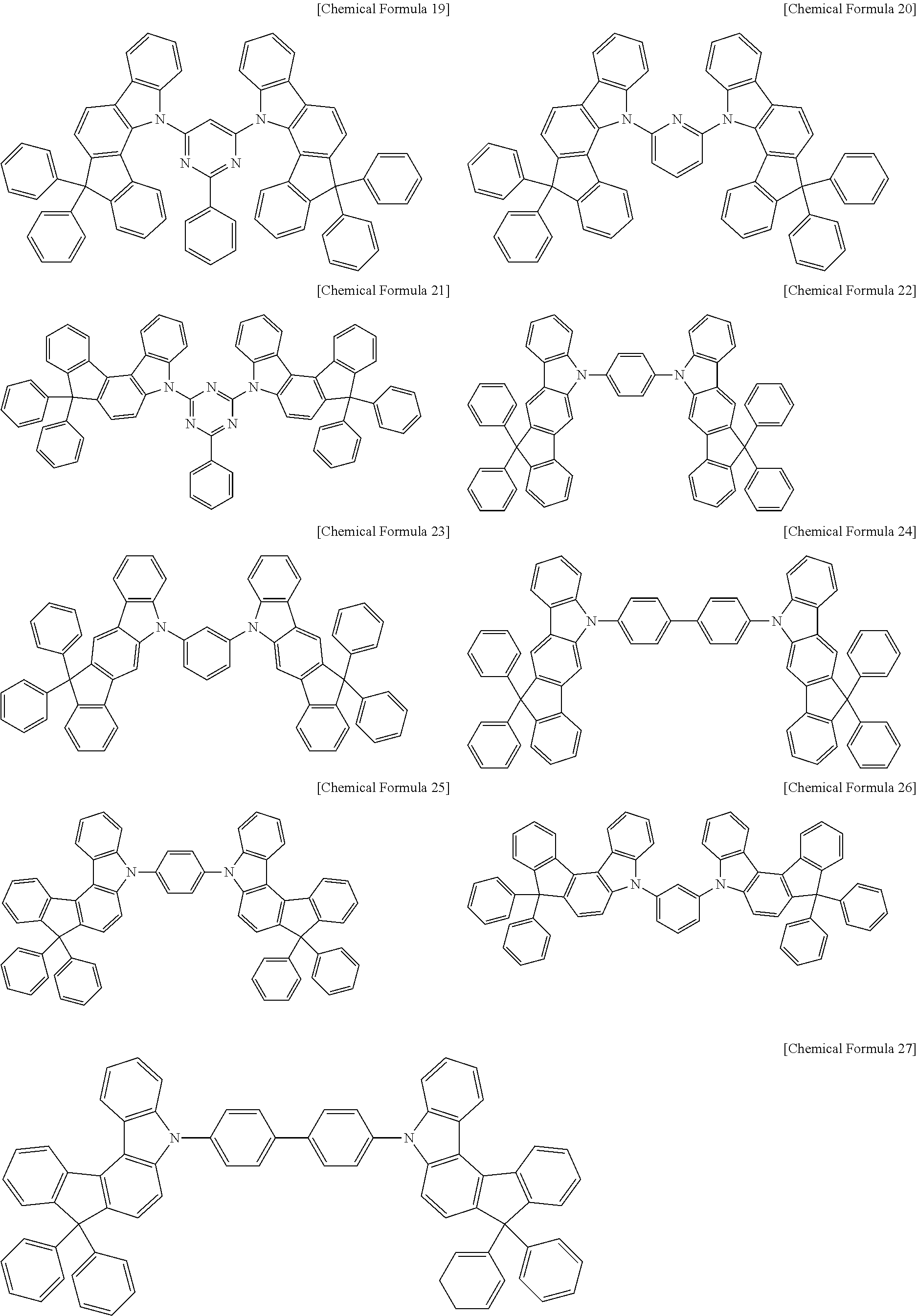

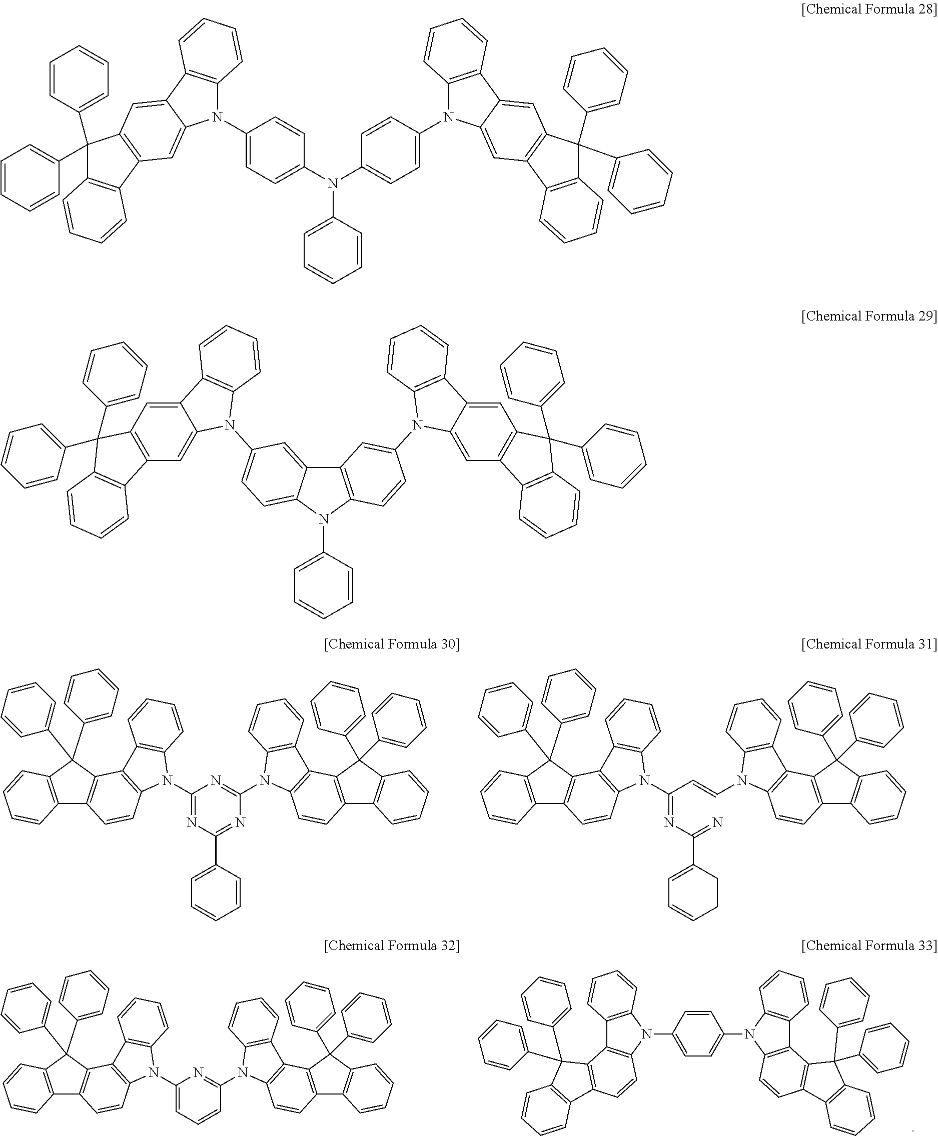

The compound for an organic optoelectronic device may be a charge transport material or a host material.

The compound for an organic optoelectronic device may have a thermal decomposition temperature (Td) of about 350 to about 600° C.

The embodiments may also be realized by providing an organic light emitting diode including an anode, a cathode, and at least one organic thin layer interposed between the anode and cathode, wherein the at least one organic thin layer includes the compound for an organic optoelectronic device according to an embodiment.

The at least one organic thin layer may include an emission layer, a hole blocking layer, an electron transport layer (ETL), an electron injection layer (EIL), a hole injection layer (HIL), a hole transport layer (HTL), an electron blocking layer, or a combination thereof.

The embodiments may also be realized by providing a display device including the organic light emitting diode according to an embodiment.

### BRIEF DESCRIPTION OF THE DRAWINGS

The embodiments will become apparent to those of ordinary skill in the art by describing in detail exemplary embodiments with reference to the attached drawings, in which:

FIGS. 1 to 5 illustrate cross-sectional views showing organic light emitting diodes including compounds according to various embodiments.

FIG. 6 illustrates a graph showing current density change depending on voltage of organic light emitting diodes according to Example 3 and Comparative Example 1.

FIG. 7 illustrates a graph showing luminance change depending on voltage of the organic light emitting diodes according to Example 3 and Comparative Example 1.

FIG. 8 illustrates a graph showing current efficiency change depending on luminance of the organic light emitting diodes according to Example 3 and Comparative Example 1.

FIG. 9 illustrates a graph showing electric power efficiency change depending on luminance of the organic light emitting diodes according to Example 3 and Comparative Example 1.

### DETAILED DESCRIPTION

Korean Patent Application No. 10-2009-0057236, filed on Jun. 25, 2009, in the Korean Intellectual Property Office, and entitled: “Compound for Organic Photoelectric Device and Organic Photoelectric Device Including the Same,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. In addition, it will also be understood that when a layer is referred to as being “between” two layers, it can be the only layer between the two layers, or one or more intervening layers may also be present. Like reference numerals refer to like elements throughout.

In the present specification, the term “substituted”, when a definition is not otherwise provided, may refer to one substituted with a halogen group, a cyano group, a C1 to C30 alkyl group, a C3 to C30 cycloalkyl group, a C6 to C30 aryl group, a C1 to C30 alkoxy group, or a combination thereof.

In the present specification the term “halogen group”, when a definition is not otherwise provided, may refer to a fluoro group, a chloro group, a bromo group, or a combination thereof.

In the present specification, the term “hetero”, when a definition is not otherwise provided, may refer to one including 1 to 3 of N, P, S, O, and remaining carbons.

In the present specification, the term “combination thereof”, when a definition is not otherwise provided, may refer to at least two substituents bound to each other by a single bond, or at least two substituents condensed to each other.

An embodiment provides a compound for an organic optoelectronic device including substituents represented by the following Chemical Formulae 1 and 2.

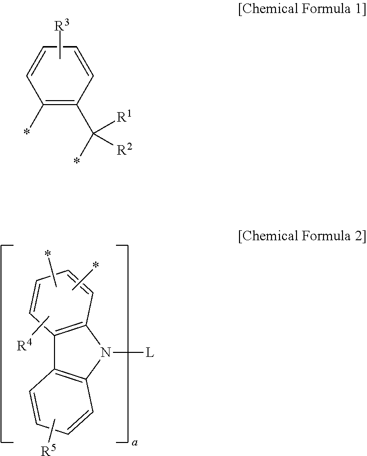

In Chemical Formulae 1 and 2, L may be a divalent to heptavalent linking group, the divalent to heptavalent linking group including, e.g., an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof. Examples of the heterocycloalkylene group may include pyrrolidine, tetrahydrofuran, tetrahydrothiophene, dioxane, dithiane, and the like. Examples of the heteroarylene group may include thiophene, furan, pyrrole, imidazole, thiazole, oxazole, oxadiazole, thiadiazole, triazole, triazine, pyridine, pyrimidine, pyridazine, pyrazine, quinoline, isoquinoline, and the like. The “combination thereof” may refer to a substituent such as carbazole, indolocarbazole, fluorene, fluorenone, benzofuran, benzothiophene, dibenzofuran, dibenzothiophene, oxanthrene, thianethrene, and the like. However, the L is not limited thereto. In Chemical Formulae 1 and 2, each * may represent an attachment point with a * of the other Chemical Formula. For example, the two *s of Chemical Formula 1 may be attached at the two *s of Chemical Formula 2.

R¹ to R⁵ may each independently be hydrogen, a substituted or unsubstituted carbazolyl group, a substituted or unsubstituted C1 to C30 alkyl group, a substituted or unsubstituted C6 to C30 aryl group, a substituted or unsubstituted C2 to C30 heteroaryl group, a substituted or unsubstituted C6 to C30 arylamino group, or a combination thereof. In an implementation, R¹ and R² may each independently be a carbazolyl group, a C1 to C30 alkyl group, a C6 to C30 aryl group, a C2 to C30 heteroaryl group, or a combination thereof, and R³ to R⁵ may each independently be hydrogen, a C1 to C30 alkyl group, a C6 to C30 aryl group, or a combination thereof.

When R¹ to R⁵ are a C6 to C30 aryl group, the aryl group may include a phenyl group, a naphthyl group, an anthracenyl group, a phenanthrenyl group, a tetracenyl group, a pyrenyl group, a fluorenyl group, or a combination thereof. However, the aryl group is not limited thereto.

When R¹ to R⁵ are a C2 to C30 heteroaryl group, the heteroaryl group may include a thiophenyl, furanyl, pyrrolyl, imidazolyl, thiazolyl, oxazolyl, oxadiazolyl, thiadiazolyl, triazolyl, triazinyl, pyridinyl, pyrimidinyl, pyridazinyl, pyrazinyl, quinolinyl, isoquinolinyl, or a combination thereof. However, the heteroaryl group is not limited thereto.

a may be an integer of 2 to 5. In an implementation, each moiety bonded to L may be the same or different.

The substituent represented by Chemical Formula 1 may be represented by the following Chemical Formula 1a.

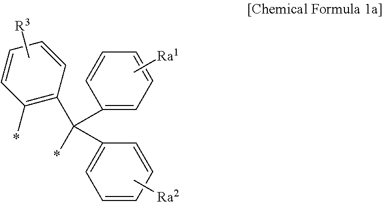

In Chemical Formula 1a, Ra¹ and Ra² may each independently be hydrogen or a C1 to C10 alkyl group, and R³ may be hydrogen, a carbazolyl group, a C1 to C30 alkyl group, a C6 to C30 aryl group, a C2 to C30 heteroaryl group, a C6 to C30 arylamino group, or a combination thereof.

The substituent represented by Chemical Formula 2 may be represented by one of the following Chemical Formulae 2a to 2c.

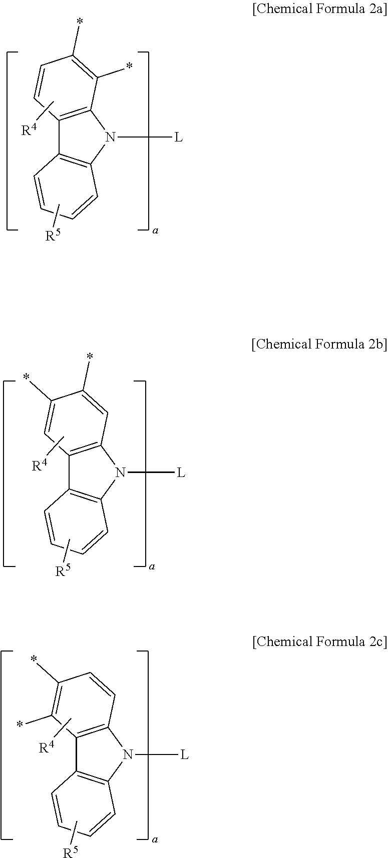

In Chemical Formulae 2a to 2c, L may be a divalent to heptavalent linking group, the divalent to heptavalent linking group including, e.g., an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof.

R⁴ and R⁵ may each independently be hydrogen, a carbazolyl group, a C1 to C30 alkyl group, a C6 to C30 aryl group, a C2 to C30 heteroaryl group, a C6 to C30 arylamino group, or a combination thereof.

a may be an integer of 2 to 5.

In an implementation, L in the above Chemical Formula 2 may be a divalent to heptavalent linking group derived from a compound represented by one of the following Chemical Formulae 2d to 2j, or a combination thereof.

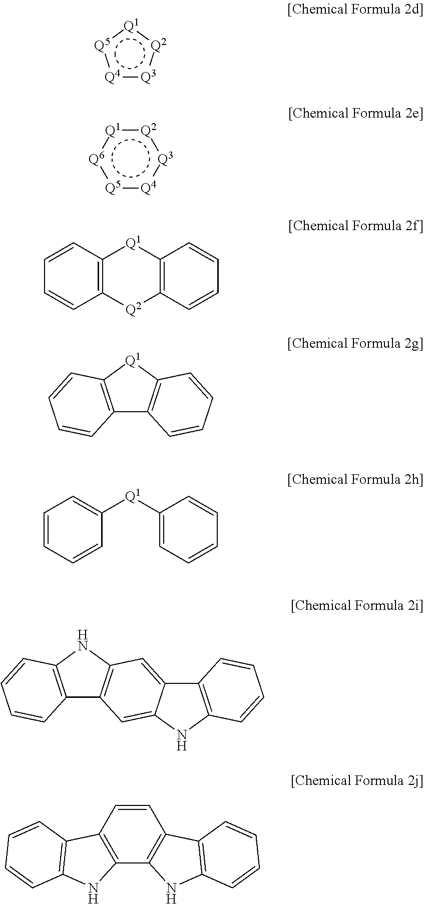

In Chemical Formulae 2d to 2j, Q¹ to Q⁶ may each independently be a substituted or unsubstituted N atom, a substituted or unsubstituted P atom, a substituted or unsubstituted S atom, a substituted or unsubstituted O atom, a substituted or unsubstituted C atom, or a combination thereof. Here, “substituted” may refer to one substituted with, e.g., an oxide group, a cyano group, a halogen group, a C1 to C30 alkyl group, a C6 to C30 aryl group, a C2 to C30 heteroaryl group, or a combination thereof.

In an implementation, in Chemical Formula 2, a may be 2 or 3.

In an implementation, L of the above Chemical Formula 2 may be represented by one of the following Formulae L-1 to L-117. However, L is not limited thereto.

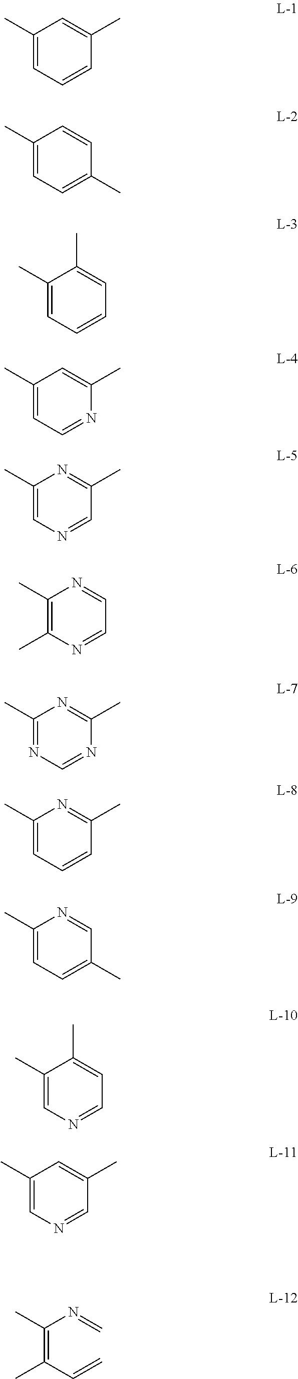

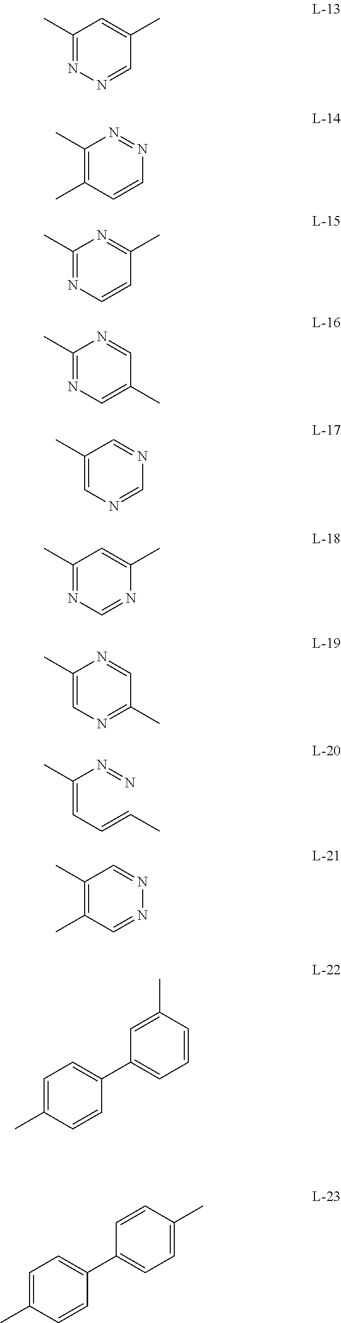

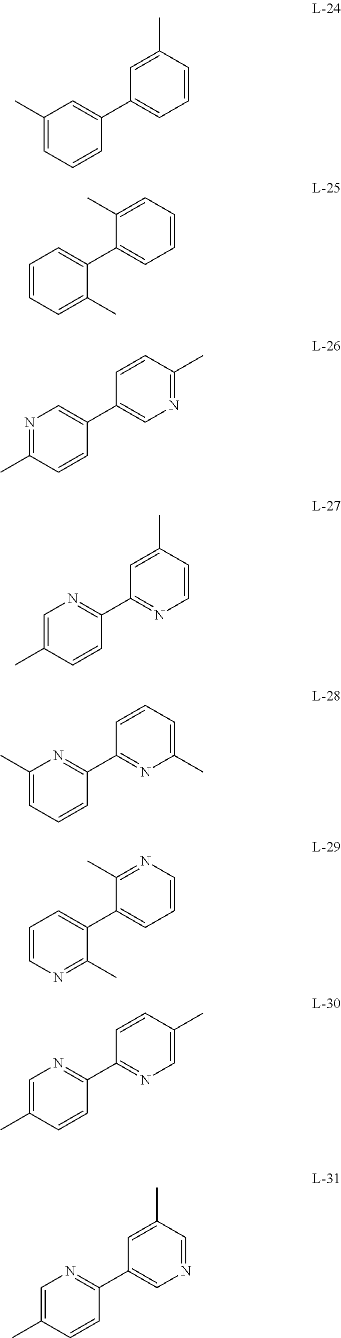

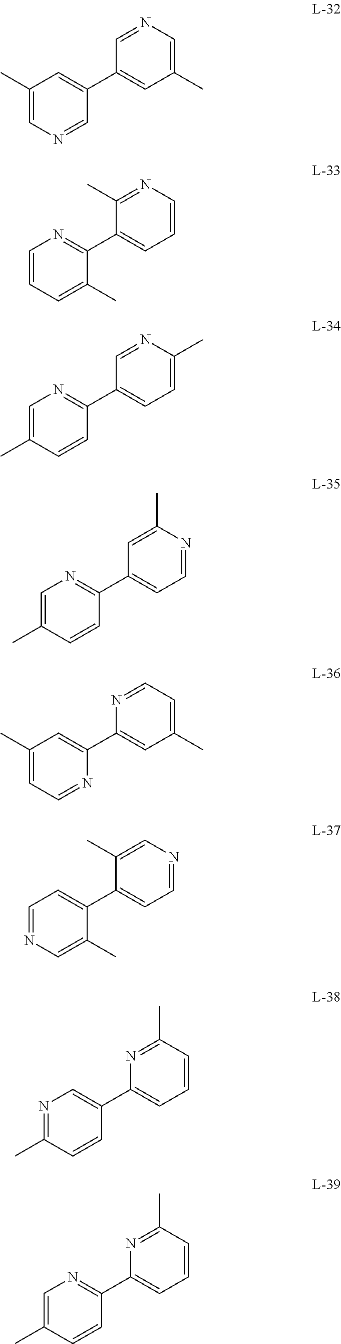

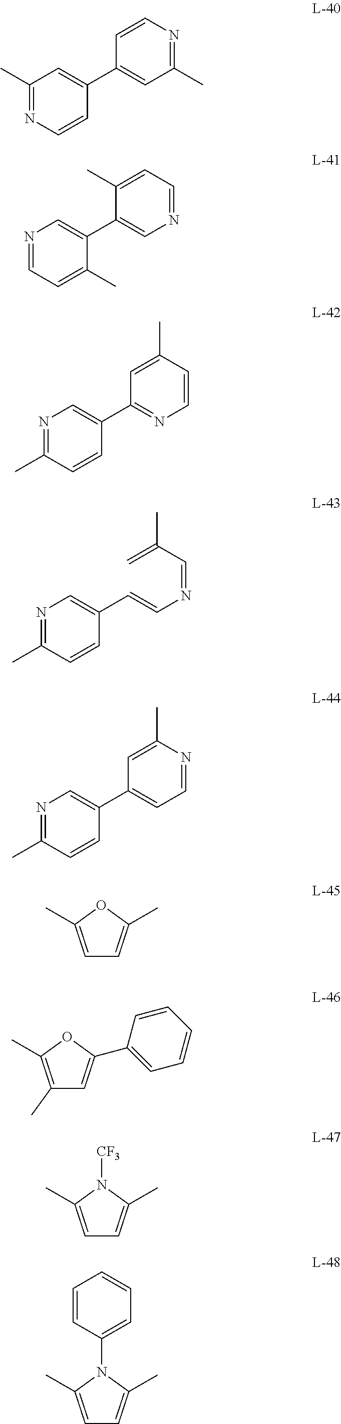

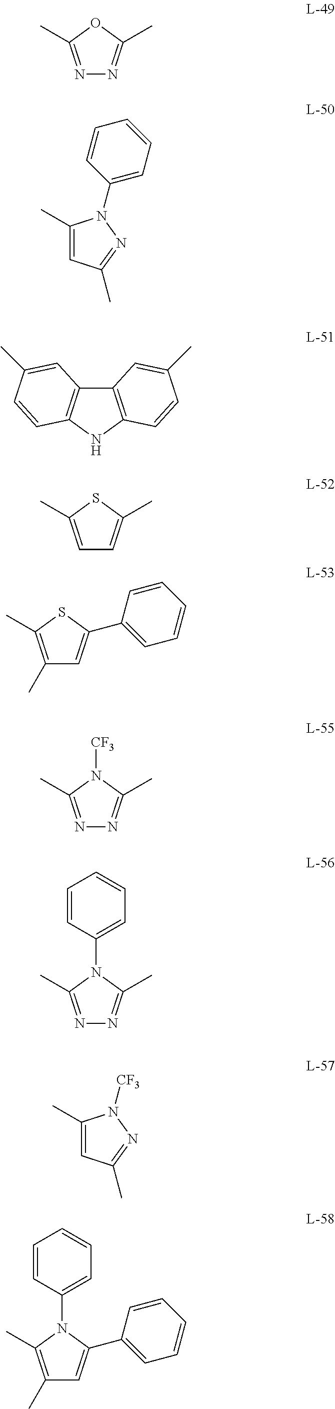

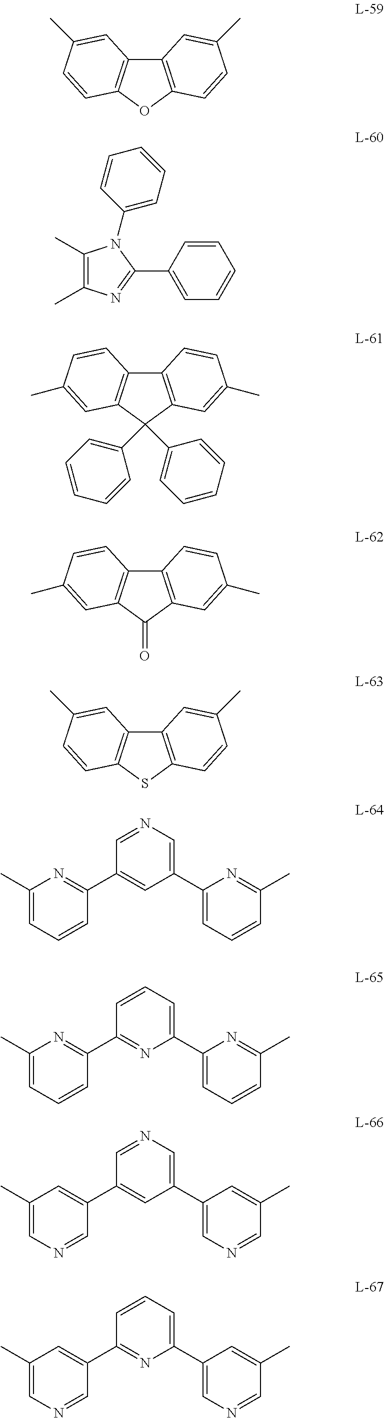

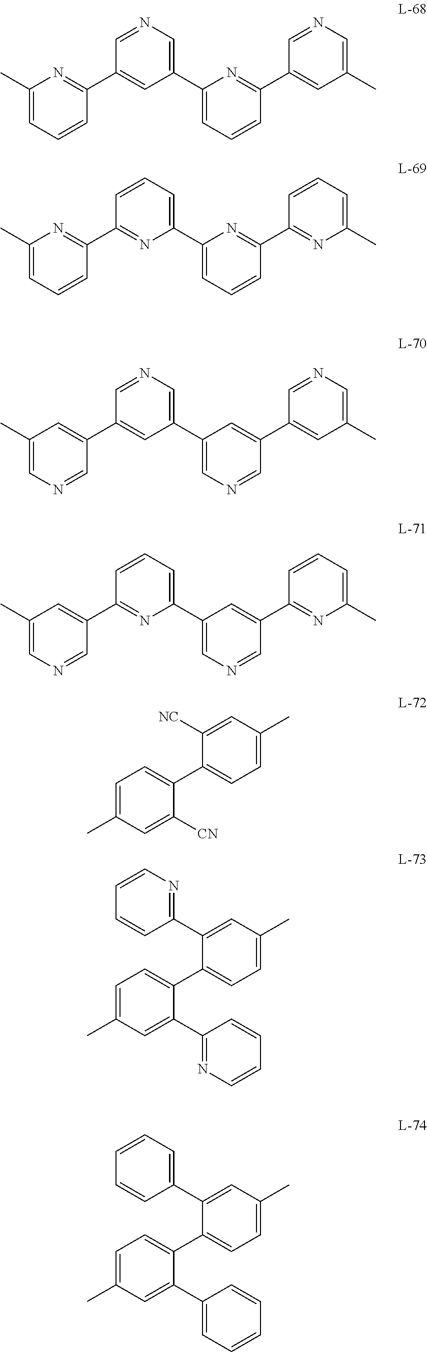

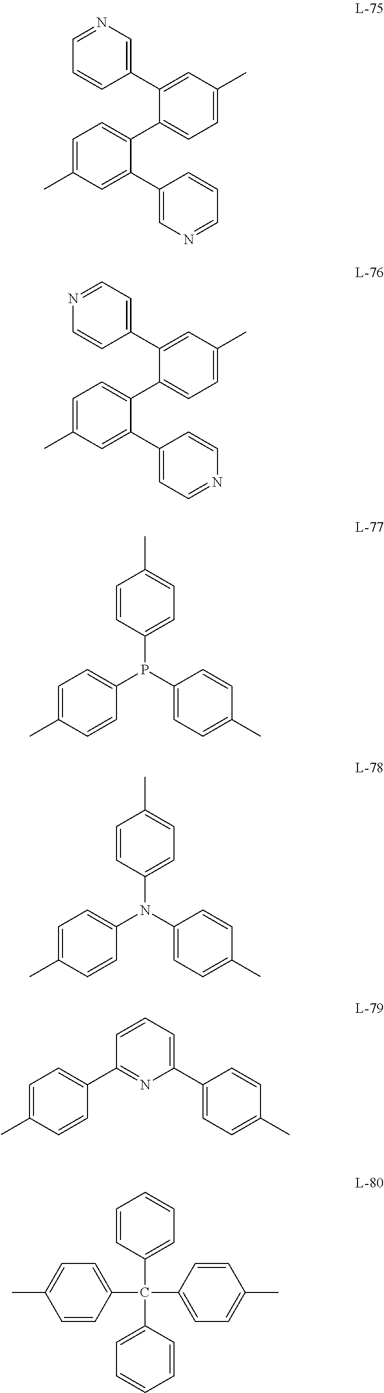

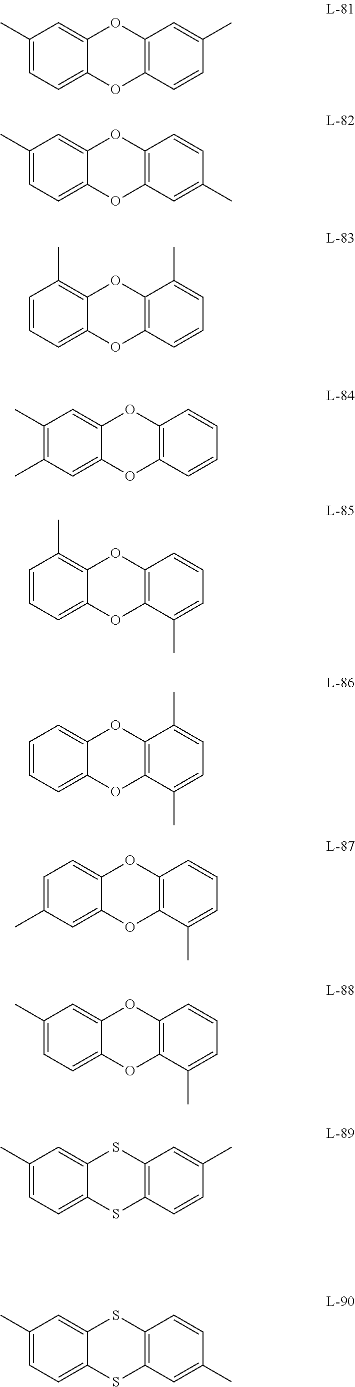

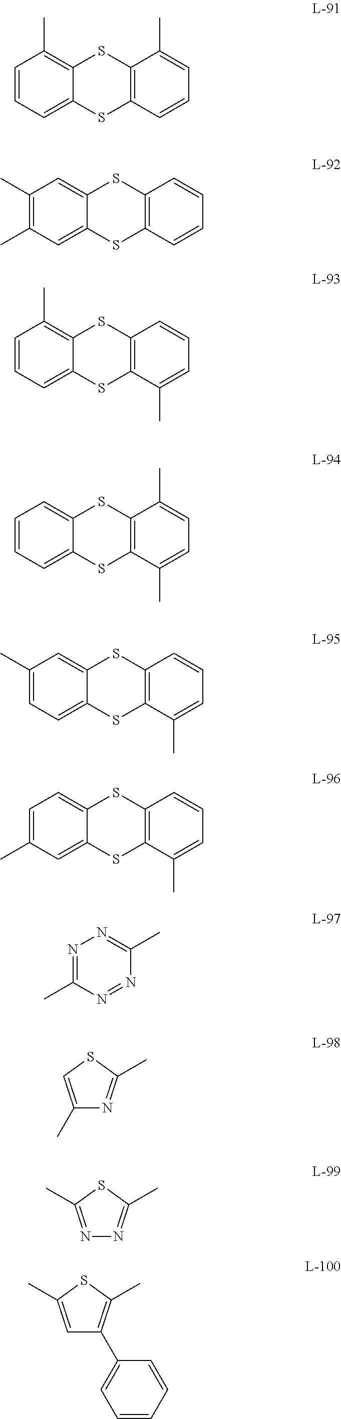

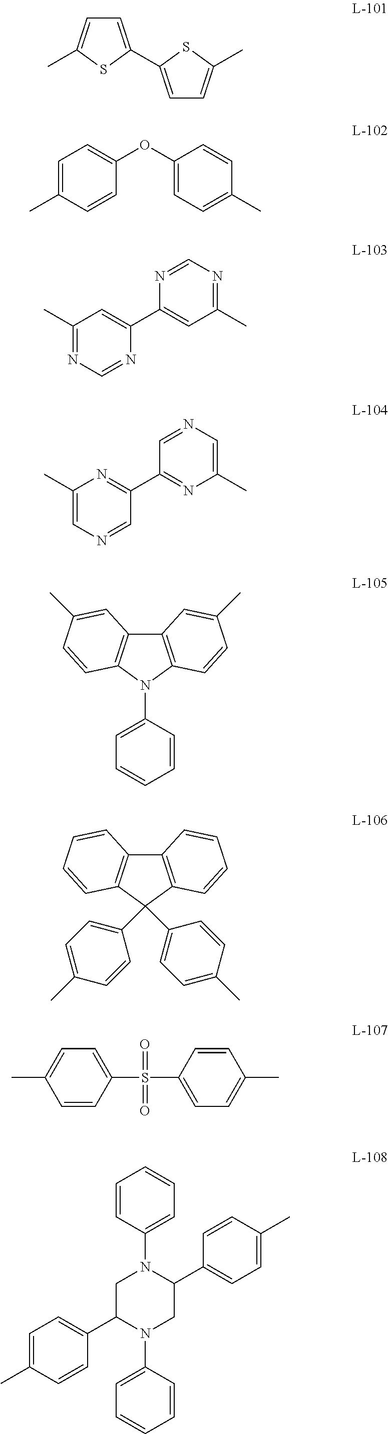

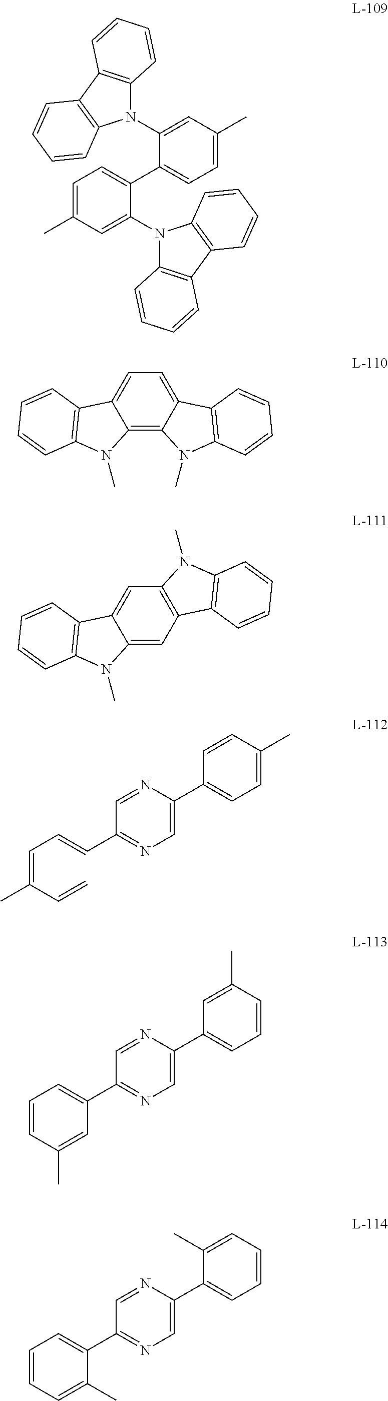

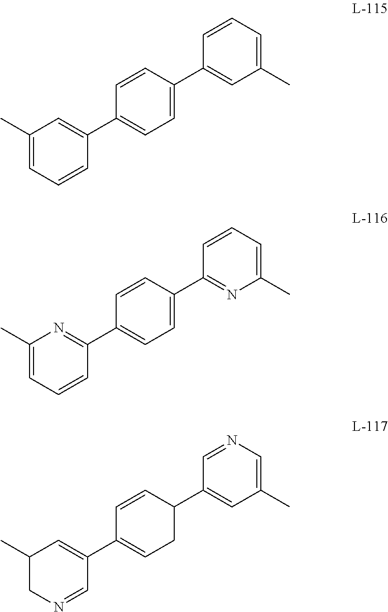

The compound for an organic optoelectronic device including the substituents represented by Chemical Formulae 1 and 2 may be represented by one of the following Chemical Formulae 3 to 8.

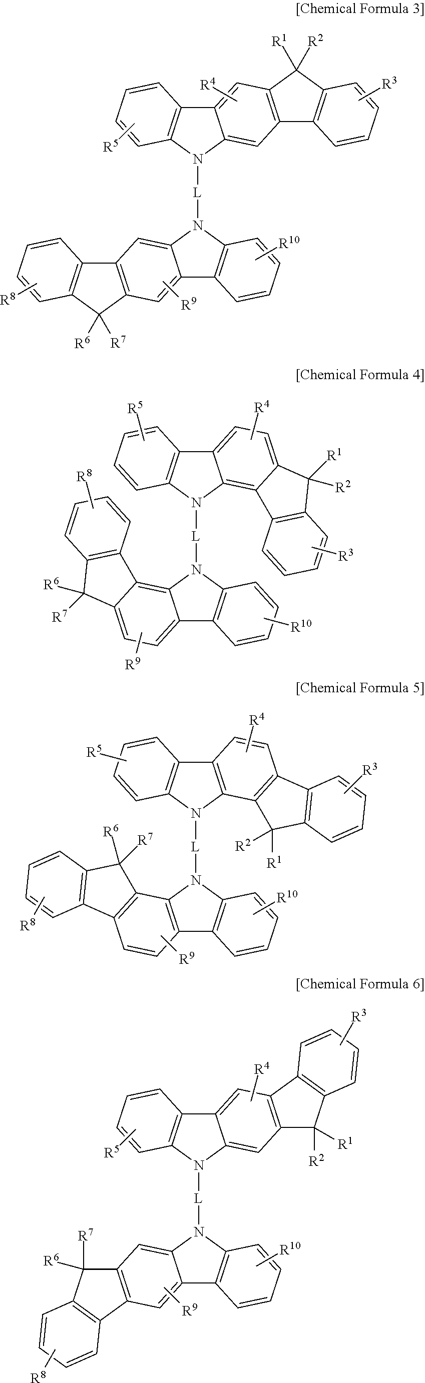

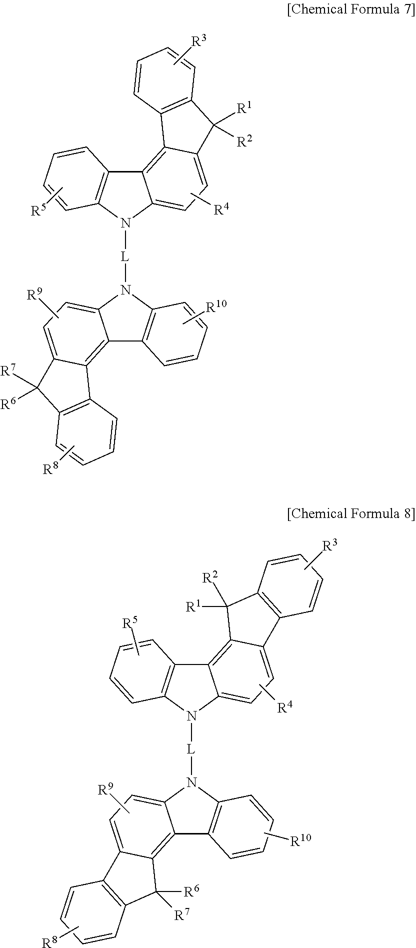

In Chemical Formulae 3 to 8, L may be a divalent linking group, and may include, e.g., an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof. R¹ to R¹⁰ may each independently be hydrogen, a substituted or unsubstituted carbazolyl group, a substituted or unsubstituted C1 to C30 alkyl group, a substituted or unsubstituted C6 to C30 aryl group, a substituted or unsubstituted C2 to C30 heteroaryl group, a substituted or unsubstituted C6 to C30 acylamino group, or a combination thereof.

The compound for an organic optoelectronic device including the substituents represented by Chemical Formulae 1 and 2 according to an embodiment may be represented by one of the following Chemical Formulae 9 to 33. However, the compound for an organic optoelectronic device is not limited thereto.

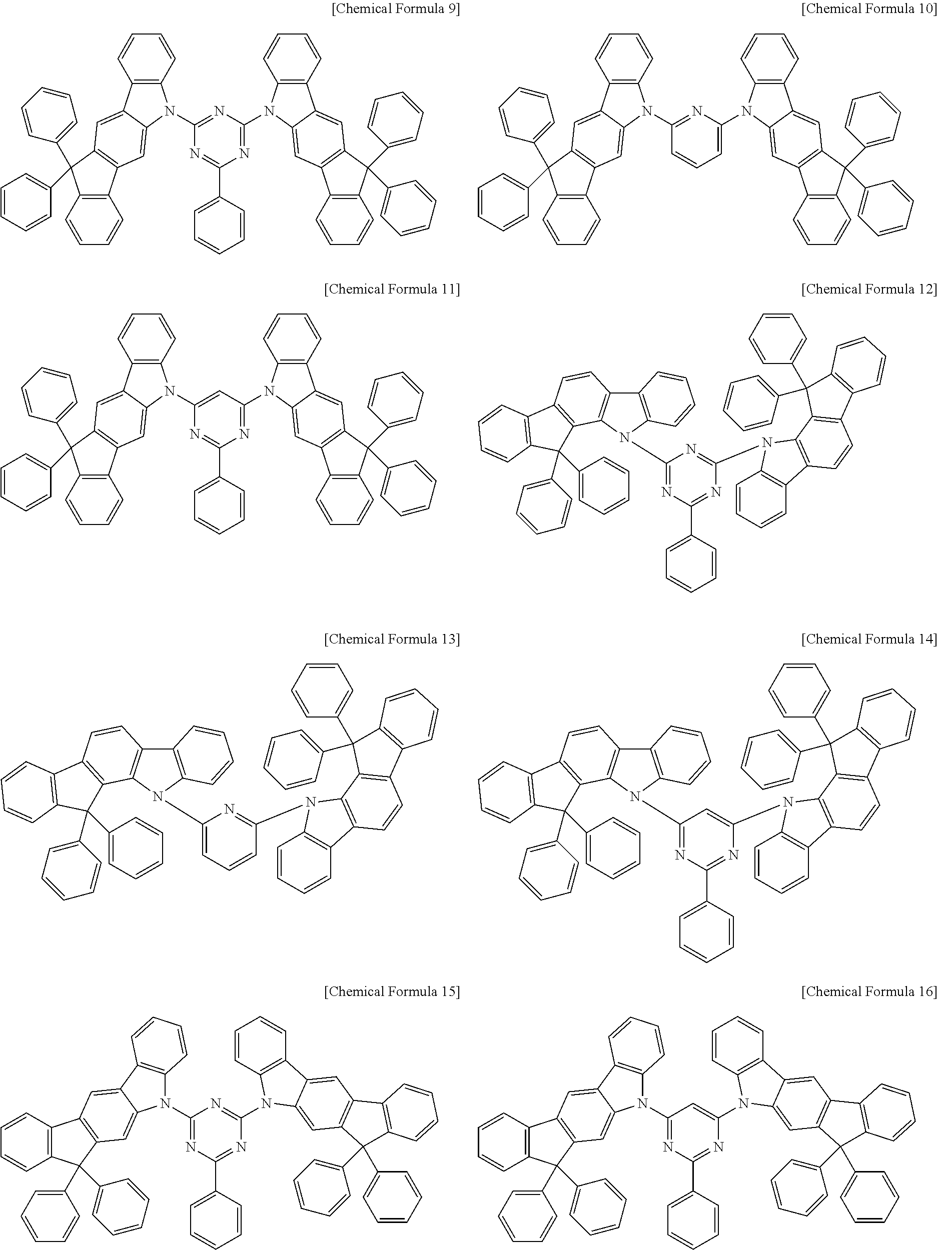

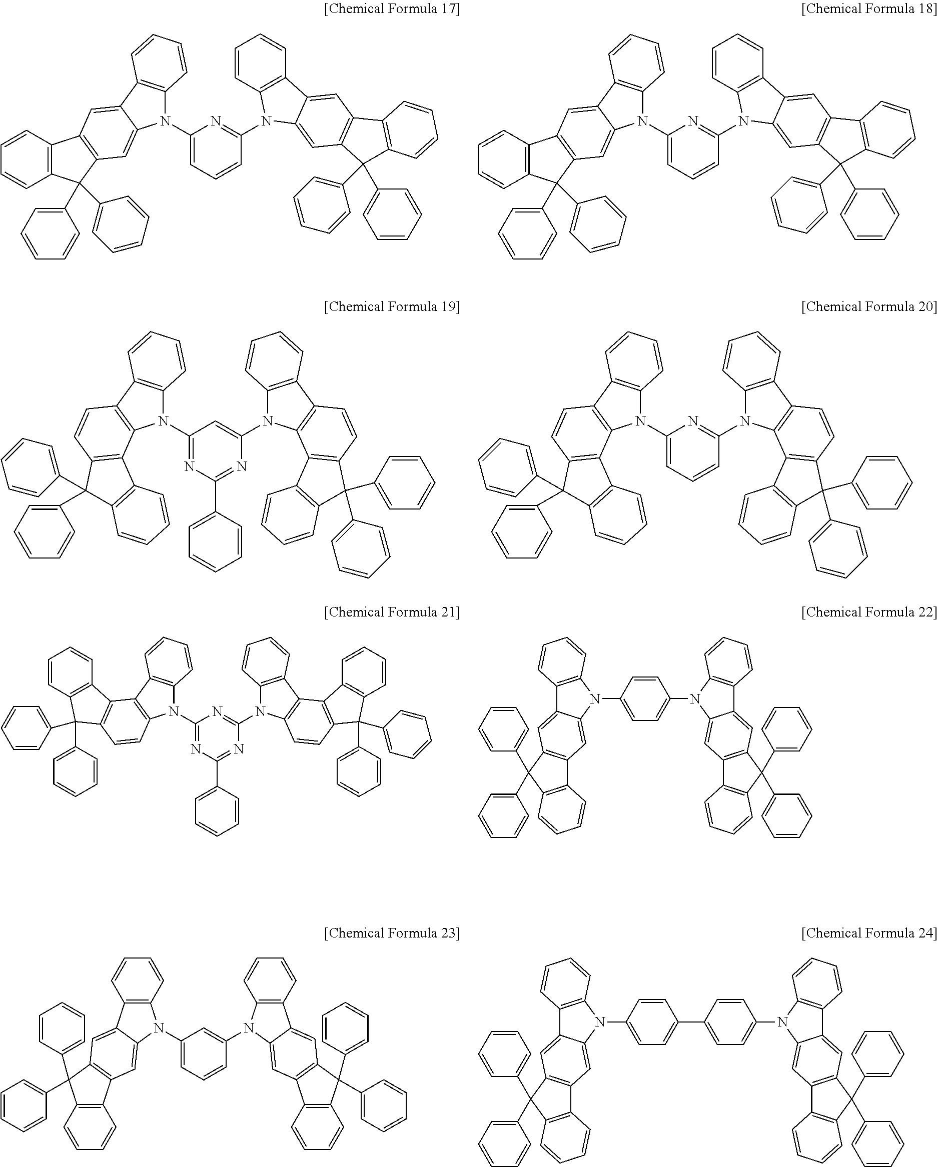

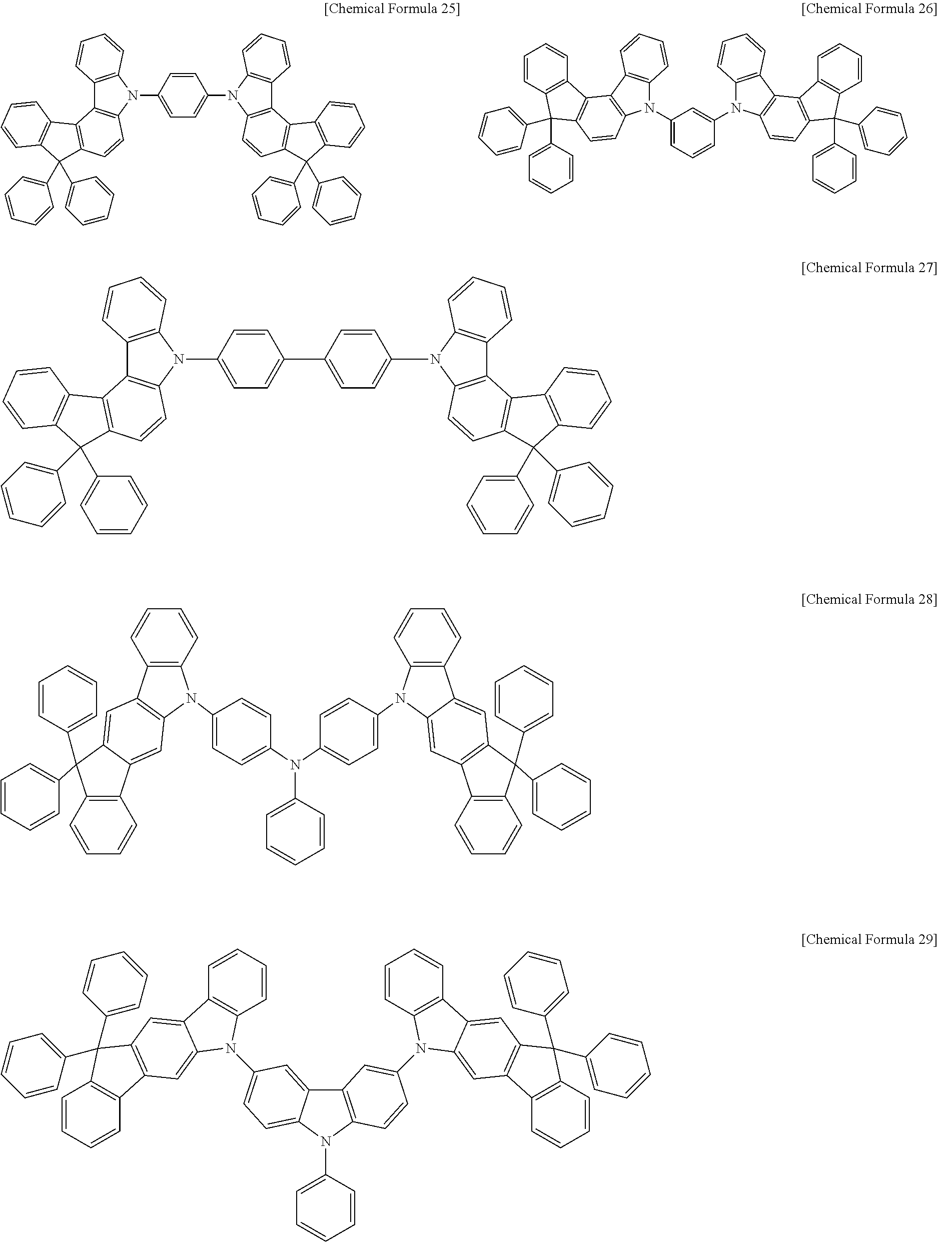

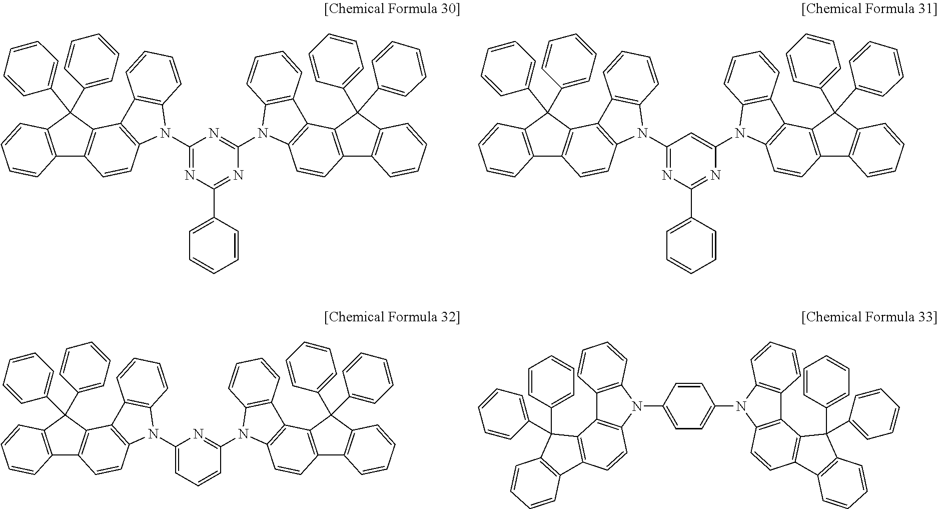

The compound for an organic optoelectronic device may be, or may be used as, a charge transport material or a host material. For example, when the compound for an organic optoelectronic device is used as a host material, the compound may be a phosphorescent host material that lowers a driving voltage and improves luminous efficiency of an organic optoelectronic device.

When the compound for an organic optoelectronic device is a host material, the compound for an organic optoelectronic device may be used as a mixture or blend with a suitable low molecular weight host material or a polymer host material. In addition, a binder resin, e.g., polyvinylcarbazole, polycarbonate, polyester, poly arylate, polystyrene, acryl polymer, methacryl polymer, polybutyral, polyvinylacetal, a diallylphthalate polymer, phenolic resin, an epoxy resin, a silicone resin, polysulfone resin, and/or a urea resin, may be mixed therewith.

For example, the low molecular weight host material may include a compound represented by one of the following Chemical Formulae 34 to 37. The polymer host material may include a polymer having a conjugated double bond, e.g., a fluorene-based polymer, a polyphenylenevinylene-based polymer, a polyparaphenylene-based polymer, or the like. However, the low molecular weight host material and polymer host material are not limited thereto.

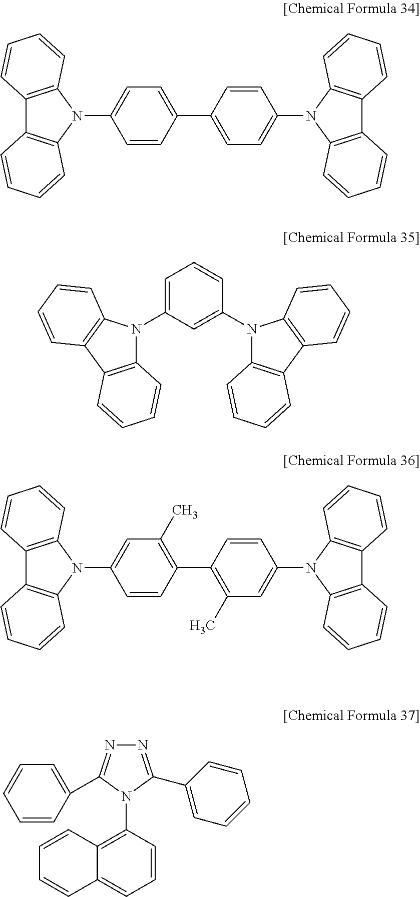

When the compound for an organic optoelectronic device is used as a host material, the compound for an organic optoelectronic device may be used singularly, or along with a dopant. The dopant may be a compound having a high emission property, by itself. However, the dopant may be added to the host in a minor amount, and may also be referred to as a guest. The dopant may be a light-emitting material while being doped in a host material. In an implementation, the dopant may include, e.g., a metal complex capable of light-emitting by multiplet excitations such as triplet excitation or more. Such a dopant may include a red (R), green (G), blue (B), and/or white (W) fluorescent or phosphorescent dopant, e.g., a red, green, blue, and/or white phosphorescent dopant. The dopant may include a material that has high luminous efficiency, is not agglomerated, and is uniformly distributed in a host material.

The phosphorescent dopant may include an organic metal compound including an element, e.g., Ir, Pt, Os, Ti, Zr, Hf, Eu, Tb, Tm, Fe, Co, Ni, Ru, Rh, Pd, or a combination thereof. For example, a red phosphorescent dopant may include platinum-octaethylporphyrin complex (PtOEP), Ir(btp)₂(acac) (bis(2-(2′-benzothienyl)-pyridinato-N,C3′)iridium(acetylacetonate)), Ir(Piq)₂(acac), Ir(Piq)₃, RD61 (UDC), and the like. A green phosphorescent dopant may include Ir(PPy)₂(acac), Ir(PPy)₃, GD48 (UDC), and the like. A blue phosphorescent dopant may include (4,6-F₂PPy)₂Irpic, fIrpic(Ir bis[4,6-di-fluorophenyl)-pyridinato-N,C2′]picolinate), and the like. The “Piq” denotes 1-phenylisoquinoline, “acac” denotes acetylacetonate, and PPy denotes 2-phenylpyridine.

The compound for an organic optoelectronic device according to an embodiment may have a thermal decomposition temperature (Td) of about 350 to about 600° C. Maintaining the Td of the compound at about 350 to about 600° C. may help ensure that the compound has excellent thermal stability and thus may be used as a host material or a charge transport material. Therefore, life-span of the organic optoelectronic device may be improved.

Another embodiment provides an organic optoelectronic device including an anode, a cathode, and an organic thin layer between the anode and the cathode, in which the organic thin layer includes the compound for an organic photoelectric device according to an embodiment. The organic optoelectronic device may include an organic photoelectronic device, an organic light emitting diode, an organic solar cell, an organic transistor, organic photo conductor drum, an organic memory device, and the like. In an organic solar cell, the compound for an organic optoelectronic device according to an embodiment may be applied to an electrode or an electrode buffer layer of an organic solar cell to improve quantum efficiency. In an implementation, the compound may be applied to an electrode material of a gate, source-drain electrodes, and the like, of an organic transistor.

The organic thin layer including the compound for an organic optoelectronic device may include an emission layer, a hole blocking layer, an electron transport layer (ETL), an electron injection layer (EIL), a hole injection layer (HIL), a hole transport layer (HTL), an electron blocking layer, or a combination thereof.

Hereinafter, an organic light emitting diode is illustrated in more detail.

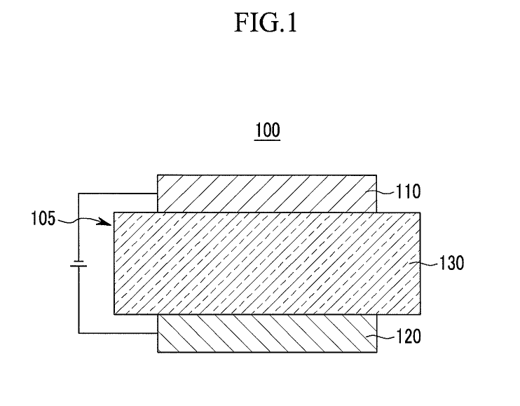

FIGS. 1 to 5 illustrate cross-sectional views showing organic light emitting diodes including the compound for an organic photoelectric device according to an embodiment.

Referring to FIGS. 1 to 5, the organic light emitting diodes 100, 200, 300, 400, and 500 according to an embodiment may include at least one organic thin layer 105 interposed between an anode 120 and a cathode 110.

A substrate of an organic light emitting diode is not particularly limited. In an implementation, the substrate may include a glass substrate or a transparent plastic substrate having excellent transparency, surface smoothness, handling ease, and water repellency.

The anode 120 may include an anode material laving a large work function in order to facilitate hole injection into an organic thin layer. The anode material may include, e.g., a metal such as nickel, platinum, vanadium, chromium, copper, zinc, gold, or the like, or an alloy of the foregoing metals; metal oxide such as zinc oxide, indium oxide, indium tin oxide (ITO), indium zinc oxide (IZO), or the like; and/or a combined metal and oxide such as ZnO/Al, SnO₂/Sb, or the like. However, the anode material is not limited thereto. In an implementation, the anode may include a transparent electrode including ITO.

The cathode 110 may include a cathode material having a small work function in order to facilitate electron injection into an organic thin layer. The cathode material may include, e.g., a metal such as magnesium, calcium, sodium, potassium, titanium, indium, yttrium, lithium, gadolinium, aluminum, silver, tin, lead, cesium, barium, or the like, or alloys thereof; or a multi-layered material such as LiF/Al, LiO₂/Al, LiF/Ca, LiF/Al, BaF₂/Ca, or the like. The cathode material is not limited thereto. In an implementation, the cathode may include a metal electrode such as aluminum.

Referring to FIG. 1, the organic light emitting diode 100 may include an organic thin layer 105 including only an emission layer 130.

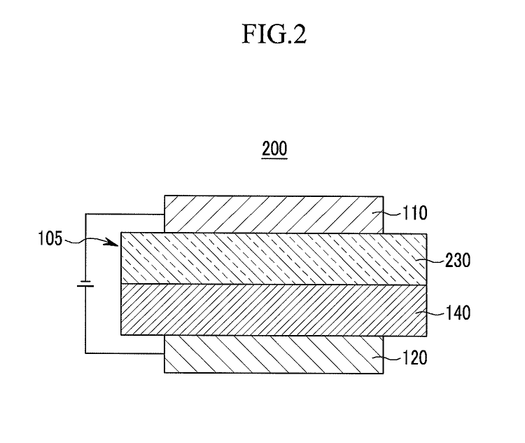

Referring to FIG. 2, a double-layered organic light emitting diode 200 may include an organic thin layer 105 including an emission layer 230 (including an electron transport layer (ETL)) and a hole transport layer (HTL) 140. The emission layer 230 may also function as an electron transport layer (ETL), and the hole transport layer (HTL) 140 layer may have excellent binding properties with a transparent electrode such as ITO (e.g., the anode 120) and/or may have excellent hole transporting properties.

The hole transport layer (HTL) 140 may include any suitable hole transport material, e.g., poly (3,4-ethylenedioxy-thiophene) (PEDOT) doped with poly(styrenesulfonate) (PSS) (PEDOT:PSS), N,N′-bis (3-methylphenyl)-N,N-diphenyl-[1,1′-biphenyl]-4,4′-diamine (TPD), N,N′-di (1-naphthyl)-N,N′-diphenylbenzidine (NPB) and the like, along with the compound for an organic optoelectronic device according to an embodiment. However, the hole transport material is not limited thereto.

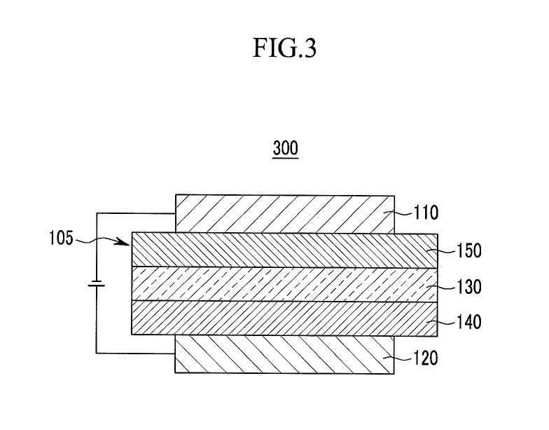

Referring to FIG. 3, a three-layered organic light emitting diode 300 may include an organic thin layer 105 including an electron transport layer (ETL) 150, an emission layer 130, and a hole transport layer (HTL) 140. The emission layer 130 may be independently installed, and layers having excellent electron transporting properties and excellent hole transporting properties may be separately stacked.

The electron transport layer (ETL) 150 may include any suitable electron transport material, e.g., aluminum tris(8-hydroxyquinoline) (Alq₃); a 1,3,4-oxadiazole derivative such as 2-(4-biphenyl-5-phenyl-1,3,4-oxadiazole (PBD); a quinoxalin derivative such as 1,3,4-tris[(3-phenyl-6-trifluoromethyl) quinoxalin-2-yl]benzene (TPQ); and a triazole derivative, along with the compound for an organic optoelectronic device according to an embodiment. However, the electron transport material is not limited thereto.

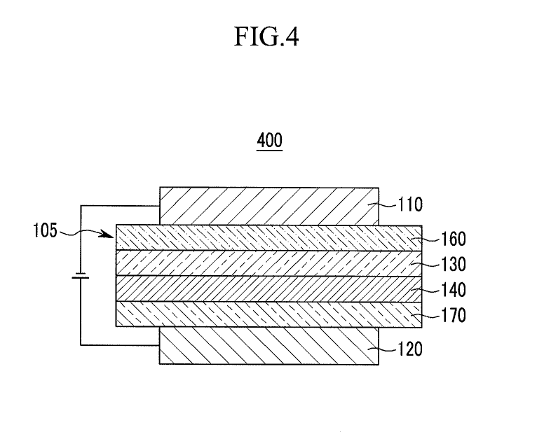

FIG. 4 illustrates a four-layered organic light emitting diode 400 that includes an organic thin layer 105 including an electron injection layer (EIL) 160, an emission layer 130, a hole transport layer (HTL) 140, and a hole injection layer (HIL) 170 (for binding with the anode 120 of ITO).

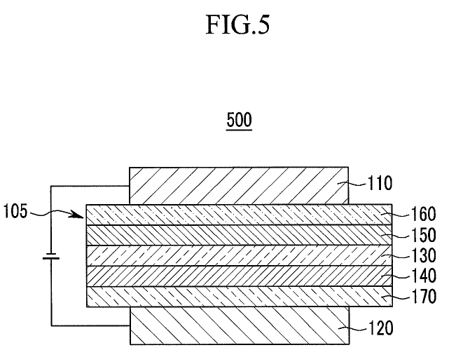

FIG. 5 illustrates a five layered organic light emitting diode 500 that includes an organic thin layer 105 including an electron transport layer (ETL) 150, an emission layer 130, a hole transport layer (HTL) 140, and a hole injection layer (HIL) 170, and further includes an electron injection layer (EIL) 160 to achieve a low voltage.

The emission layers 130 and 230 may have a thickness of about 5 to about 1,000 nm, and the hole transport layer (HTL) 140 and electron transport layer (ETL) 150 may each have a thickness of about 10 to about 10,000 Å. However, the thicknesses are not limited thereto.

In FIGS. 1 to 5, the organic thin layer 105 (selected from the electron transport layer (ETL) 150, electron injection layer (EIL) 160, emission layer 130 and 230, hole transport layer (HTL) 140, hole injection layer (HIL) 170, and/or a combination thereof) may include the compound for an organic optoelectronic device according to an embodiment. The compound for an organic optoelectronic device may be used for an electron transport layer (ETL) 150, a hole transport layer (HTL) 140, and/or electron injection layer (EIL) 160. When the compound is used for the electron transport layer (ETL), it is possible to provide an organic light emitting diode having a simpler structure because an additional or separate hole blocking layer may be omitted.

Furthermore, when the compound for an organic optoelectronic device is included in the emission layer 130 and 230, the compound for an organic optoelectronic device may be included as a phosphorescent host, and the emission layer 130 and 230 may further include a dopant. In an implementation, the dopant may include a red, green, blue, and/or white phosphorescent dopant.

The organic light emitting diode may be fabricated by: forming an anode on a substrate, forming an organic thin layer (by a dry coating method such as evaporation, sputtering, plasma plating, and ion plating, or a wet coating method such as spin coating, dipping, and flow coating); and providing a cathode thereon.

Another embodiment provides a display device including the organic light emitting diode.

The following Examples and Comparative Examples are provided in order to set forth particular details of one or more embodiments. However, it will be understood that the embodiments are not limited to the particular details described. Further, the Comparative Examples are set forth to highlight certain characteristics of certain embodiments, and are not to be construed as either limiting the scope of the invention as exemplified in the Examples or as necessarily being outside the scope of the invention in every respect.

Synthesis of Compound for Organic Optoelectronic Device

### Example 1

A compound for an organic optoelectronic device was synthesized according to the following Reaction Scheme 1.

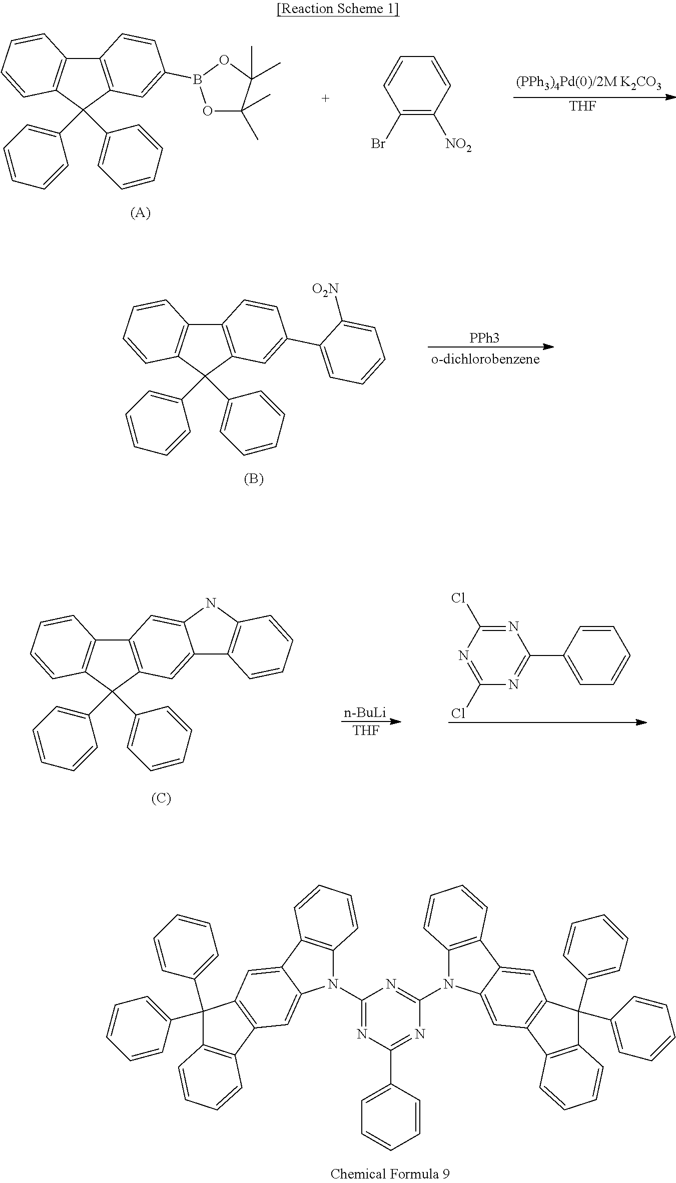

First Step: Synthesis of Intermediate Product (B)

11.0 g (24.7 mmol) of a compound A, 6.0 g (29.7 mmol) of 1-bromo-2-nitro benzene, and 1 g (0.86 mmol) of tetrakis(triphenylphosphine)palladium were mixed with 200 mL of tetrahydrofuran (THF) in a 500 mL round-bottomed flask with a thermometer, a reflux-condenser, and an agitator under an argon atmosphere, and 250 mL of 2M potassium carbonate was added thereto. The resulting mixture was agitated at 75° C. for 24 hours.

The reactant was cooled down to complete the reaction, extracted with methylene chloride, and cleaned with water. Next, the resulting reactant was treated with anhydrous magnesium sulfate to remove moisture therefrom and filtered to remove an organic solvent. The final residue was purified through silica gel chromatography using a mixed solvent prepared by mixing methylene chloride and hexane in a volume ratio of 1:1, obtaining 9 g of an intermediate product (B) (yield: 82.7%).

Second Step: Synthesis of Intermediate Product (C)

8 g (18.2 mmol) of the intermediate product (B) (synthesized in the first step) and 14.3 g (54.6 mmol) of triphenylphosphine were dissolved in 150 ml of dichlorobenzene, and the solution was heated and refluxed at 160° C. under an argon atmosphere.

The organic solvent was distilled and removed under reduced pressure, extracted with methylene chloride, and cleaned with water. Then, the reactant was treated with anhydrous magnesium sulfate to remove moisture therefrom and filtered to remove remaining organic solvent. The final residue was purified through silica gel chromatography using a mixed solvent prepared by mixing methylene chloride and hexane in a volume ratio of 2:1, obtaining 5.3 g of an intermediate product (C) (yield: 71.5%).

Third Step: Synthesis of Compound for Organic Optoelectronic Device

5 g (12.2 mmol) of the intermediate product (C) (synthesized in the second step) was dissolved in 100 mL of anhydrous tetrahydrofuran (THF), and 9.2 mL of 1.6 M n-BuLi was slowly added thereto in a dropwise fashion at −78° C. The mixture was agitated for 30 minutes. Subsequently, the mixture was further agitated at room temperature (˜25° C.) for 20 minutes and mixed with 1.32 g (5.8 mmol) of 2,4-dichloro-6-phenyl triazine at −78° C. The resulting mixture was agitated at a room temperature for 12 hours.

Then, the reactant was brought to room temperature to complete the reaction, extracted with methylene chloride, and washed with water. The resulting reactant was treated with anhydrous magnesium sulfate to remove moisture and filtered to remove organic solvent therefrom. The final residue was purified and recrystallized through silica gel chromatography using a mixed solvent prepared by mixing methylene chloride and hexane in a volume ratio of 1:3, obtaining 2.7 g of a compound (Chemical Formula 9) for an organic optoelectronic device (yield: 48.0%).

Atomic analysis was performed on the compound for an organic optoelectronic device. The result is provided as follows;

Calcd. C, 88.08; H, 4.68; N, 7.23.

Found. C, 88.10; H, 4.66; N, 7.23.

### Example 2

A compound for an organic optoelectronic device was synthesized according to the following Reaction Scheme 2.

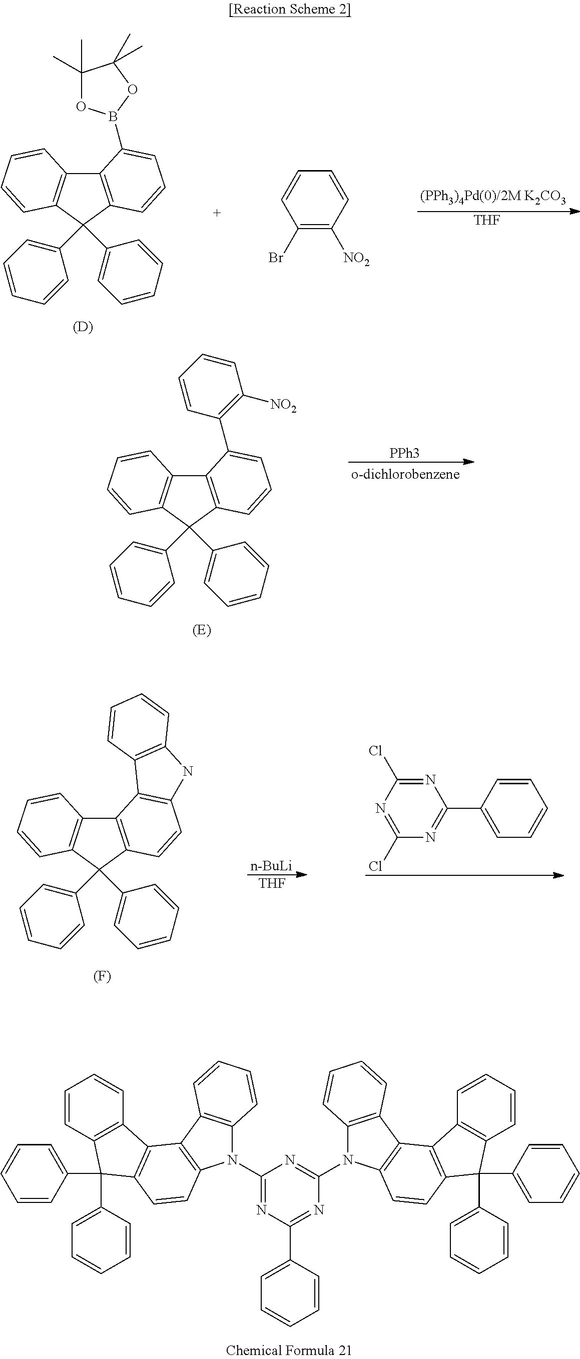

First Step: Synthesis of Intermediate Product (E)

10.0 g (22.5 mmol) of a compound D, 5.45 g (27.0 mmol) of 1-bromo-2-nitro benzene, and 1 g (0.86 mmol) of tetrakis(triphenylphosphine)palladium were dissolved in 200 mL of tetrahydrofuran in a 500 mL round-bottomed flask with a thermometer, a reflux-condenser, and an agitator under an argon atmosphere, and 50 mL of tetratriethyl ammonium hydroxide with 20% of a concentration was added thereto. The mixture was agitated at 75° C. for 24 hours.

Next, the reactant was cooled down to complete the reaction, extracted with methylene chloride, and washed with water. Then, the resulting reactant was treated with anhydrous magnesium sulfate to remove moisture therefrom and filtered to remove the organic solvent. The final residue was purified through silica gel chromatography using a mixed solvent prepared by mixing methylene chloride and hexane in a volume ratio of 1:1, obtaining 7.8 g of an intermediate product (E) (yield: 78.8%).

Second Step: Synthesis of Intermediate Product (F)

7 g (15.9 mmol) of the intermediate product (E) (synthesized in the first step) and 14.3 g (54.6 mmol) of triphenylphosphine were dissolved in 150 ml of dichlorobenzene, and the solution was heated and refluxed at 160° C. under an argon atmosphere.

The resulting reactant was distilled under a reduced pressure to remove organic solvent, extracted with methylene chloride, and washed with water. Then, the reactant was treated with anhydrous magnesium sulfate to remove moisture and filtered to remove the organic solvent. The final residue was purified through silica gel chromatography using a mixed solvent prepared by mixing methylene chloride and hexane in a volume ratio of 2:1, obtaining 4.4 g of an intermediate product (F) (yield: 68%).

Third Step: Synthesis of Compound for Organic Optoelectronic Device

4 g (9.8 mmol) of the intermediate product (F) (synthesized in the second step) was dissolved in 100 mL of anhydrous tetrahydrofuran (THF), and 9.2 mL of 1.6 M n-BuLi was slowly added in a dropwise fashion thereto at −78° C. for 30 minutes. The resulting mixture was further agitated at room temperature for 20 minutes and then, mixed with 1 g (4.4 mmol) of 2,4-dichloro-6-phenyl triazine at −78° C. The mixture was agitated at room temperature for 12 hours.

The agitated reactant was brought to room temperature to complete the reaction, extracted with methylene chloride, and washed with water. Next, the reactant was treated with anhydrous magnesium sulfate to remove moisture and filtered to remove the organic solvent. The final residue was purified and recrystallized through silica gel chromatography using a mixed solvent prepared by mixing methylene chloride and hexane in a volume ratio of 1:3, obtaining 1.8 g of a compound (Chemical Formula 21) for an organic optoelectronic device (yield: 42.3%).

Atomic analysis was performed on the obtained compound for an organic optoelectronic device. The result is provided as follows;

Calcd. C, 88.08; H, 4.68; N, 7.23.

Found. C, 88.06; H, 4.70; N, 7.23.

Preparation of Organic Light Emitting Diode

### Example 3

An organic light emitting diode was fabricated by using the compound synthesized in Example 1 as a host and Ir(PPy)₃ as a dopant. Herein, an ITO layer was formed to be 1,000 Å thick to serve as an anode, and an aluminum (Al) layer was formed to be 1,500 Å thick to serve as a cathode.

In particular, an anode for an organic light emitting diode was fabricated by cutting an ITO glass substrate (with a sheet resistance of 15 Ω/cm²) to have a size of 50 mm×50 mm×0.7 mm and then, performing ultrasonic wave cleaning in acetone, isopropyl alcohol, and pure water, respectively, for 15 minutes and UV ozone-cleaning for 30 minutes.

On the substrate, a 800 Å hole transport layer (HTL) was formed by depositing N,N′-di(1-naphthyl)-N,N′-diphenylbenzidine (NPB) (70 nm) and 4,4′,4″-tri(N-carbazolyl)triphenylamine (TCTA) (10 nm) with a vacuum degree of 650×10⁻⁷ Pa at a deposition speed ranging from 0.1 to 0.3 nm/s.

Next, the compound synthesized according to Example 1 was deposited to form a 300 Å-thick emission layer under the same vacuum deposition conditions described above, and Ir(PPy)₃ as a phosphorescent dopant was simultaneously deposited. Herein, the phosphorescent dopant was regulated regarding deposition speed to be deposited in an amount of 10 wt %, based on 100 wt % of the total weight of the emission layer.

On the emission layer, a 50 Å-thick hole-blocking layer was formed by depositing aluminum(III) bis(2-methyl-8-quinolinato)4-phenylphenolate (BAlq) under the same vacuum deposition conditions described above.

Then, a 200 Å-thick electron transport layer (ETL) was formed thereon by depositing Alq₃ under the same vacuum deposition conditions described above.

On the electron transport layer (ETL), LiF and Al were sequentially deposited to form a cathode, fabricating an organic light emitting diode.

The organic light emitting diode had a structure of ITO/NPB (70 nm)/TCTA (10 nm)/EML (the compound of Example 1 (90 wt %)+Ir(PPy)₃ (10 wt %), 30 nm)/Balq (5 nm)/Alq₃ (20 nm)/LiF (0.5 nm)/Al (150 nm).

### Comparative Example 1

An organic light emitting diode was fabricated according to the same method as Example 3 except for using 4,4-N,N-dicarbazolebiphenyl (CBP) as a host for an emission layer, instead of the compound synthesized according to Example 1.

### Experimental Example 1

### Performance Evaluation of Organic Light Emitting Diode

The organic light emitting diodes according to Example 3 and Comparative Example 1 were measured regarding current density change and luminance change depending on a voltage and luminous efficiency. In particular, the measurement was performed as follows. The result is provided in the following Table 1.

(1) Current Density Change Depending on Voltage Change

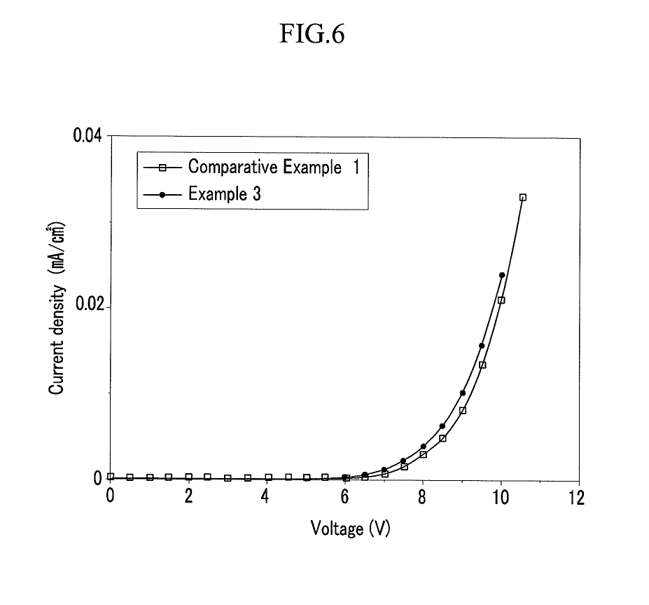

The organic light emitting diodes were measured regarding current using a current-voltage meter (Keithley 2400) while their voltages were increased from 0 V to 10 V. The current values were divided by an area to calculate current density. The results are provided in FIG. 6.

(2) Luminance Change Depending on Voltage Change

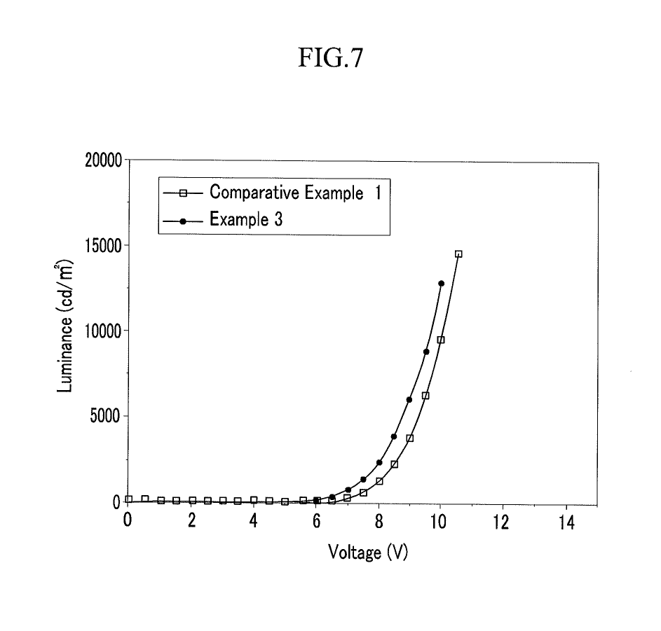

The organic light emitting diodes were measured regarding luminance using a luminance meter (Minolta Cs-1000A) while their voltages were increased from 0 V to 10 V. The results are provided in FIG. 7.

(3) Luminous Efficiency

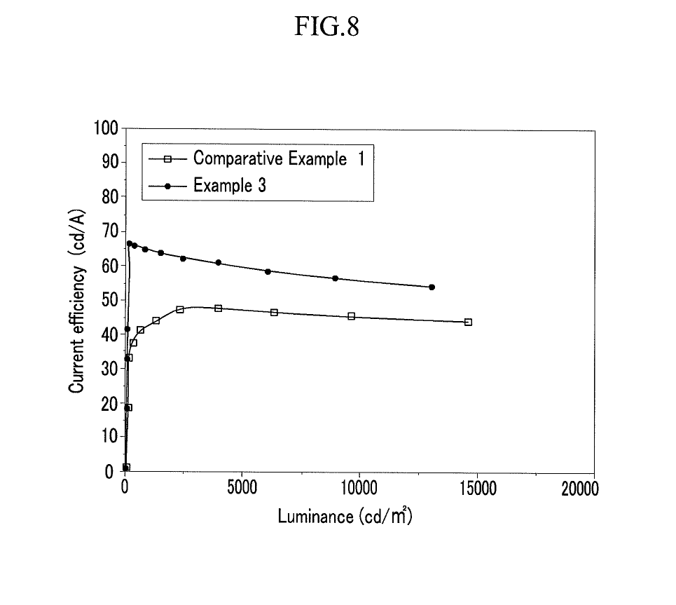

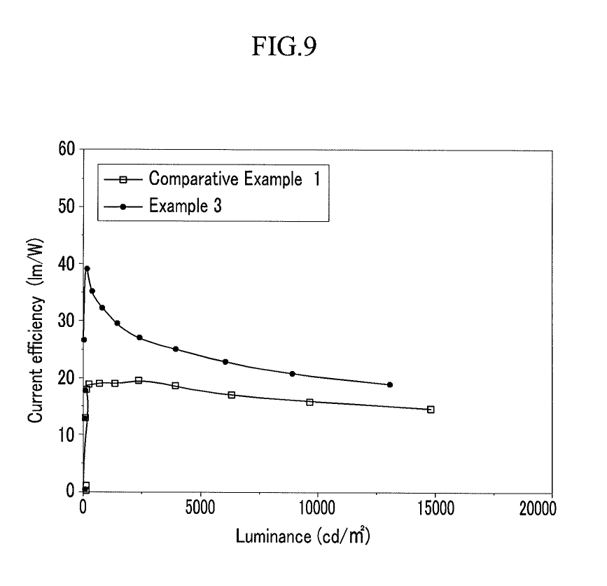

The luminance and current density obtained from the above (1) and (2) and a voltage were used to calculate current efficiency (cd/A) and electric power efficiency (lm/W) at the same luminance (2000 cd/m²). The results are provided in FIGS. 8 and 9.

(4) Color Coordinate

The organic light emitting diodes were measured regarding color coordinate using a luminance meter (Minolta Cs-100A). The results are provided in the following Table 1.

TABLE 1

| | | At 2000 cd/m² | | | |
|---|---|---|---|---|---|
| | Host | | | Electric | |
| | material of | Driving | Current | power | Color |
| | emission | voltage | efficiency | efficiency | coordinate |
| Device | layer | (V) | (cd/A) | (lm/W) | (x, y) |
| Example 3 | Example 1 | 7.7 | 63.2 | 28.4 | 0.301, 0.622 |
| Comparative | CBP | 8.3 | 46.3 | 19.1 | 0.293, 0.622 |
| Example 1 | | | | | |

Referring to Table 1, it may be seen that the organic light emitting diode of Example 3 had a low driving voltage and much improved current efficiency and electric power efficiency, compared with the organic light emitting diode of Comparative Example 1, based on the characteristic evaluation results. Without being bound by theory, it is believed that the compound of Example 1 helped to lower driving voltage of the organic light emitting diode and to improve luminance and efficiency.

By way of summation and review, a dopant (along with a host material) may be included in an emission layer to increase efficiency and stability of organic light emitting diode. 4-N,N-dicarbazolebiphenyl (CBP) has been considered as a host material. However, CBP has high structural symmetry and may be easily crystallized. Due to low thermal stability, a short-circuit or a pixel defect may occur during heat resistance test of a device. Furthermore, host materials (such as CBP) may have faster hole transport speed than electron transport speed. Thus, an exciton may not be effectively formed in an emission layer, decreasing luminous efficiency of a device.

A low molecular weight host material may be deposited using a vacuum-deposition, which may cost more than a wet process. Further, low molecular weight host materials may have low solubility in an organic solvent. Thus, they may not be applied in a wet process and may not form an organic thin layer having excellent film characteristics.

Accordingly, in order to realize an organic photoelectric device with excellent efficiency and life-span, the embodiments provide a phosphorescent host material and a charge transport material having excellent electrical and thermal stability and bipolar characteristics (effectively transporting both holes and electrons) or a host material mixed with a material being capable of effectively transporting holes and electrons.

An embodiment provides a compound for an organic optoelectronic device having excellent thermal stability, and being capable of effectively transporting both holes and electrons.

Another embodiment provides an organic optoelectronic device including the compound for an organic optoelectronic device and having excellent efficiency and a driving voltage.

The compound for an organic optoelectronic device according to an embodiment may have excellent thermal stability, and particularly, may be applied to an organic thin layer of an organic optoelectronic device and thus may provide an organic optoelectronic device and a display device having high luminous efficiency at a low voltage and improved life-span.

Example embodiments have been disclosed herein, and although specific terms are employed, they are used and are to be interpreted in a generic and descriptive sense only and not for purpose of limitation. Accordingly, it will be understood by those of skill in the art that various changes in form and details may be made without departing from the spirit and scope of the present invention as set forth in the following claims.

What is claimed is:

1. A compound for an organic optoelectronic device comprising substituents represented by the following Chemical Formulae 1 and 2: 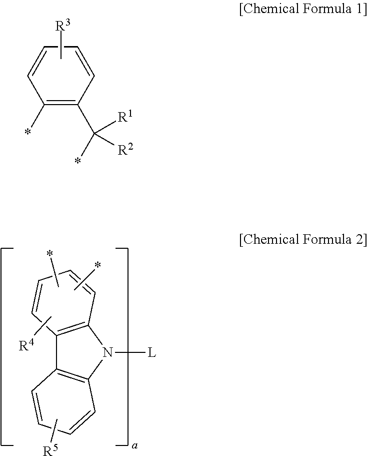
wherein, in Chemical Formulae 1 and 2:
L is a divalent to heptavalent linking group, the divalent to heptavalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof,
R¹ to R⁵ are each independently hydrogen, a substituted or unsubstituted carbazolyl group, a substituted or unsubstituted C1 to C30 alkyl group, a substituted or unsubstituted C6 to C30 aryl group, a substituted or unsubstituted C2 to C30 heteroaryl group, a substituted or unsubstituted C6 to C30 arylamine group, or a combination thereof, and
a is an integer of 2 to 5.

2. The compound for an organic optoelectronic device as claimed in claim 1, wherein R¹ and R² in Chemical Formula 1 are each independently a carbazolyl group, a C1 to C30 alkyl group, a C6 to C30 aryl group, a C2 to C30 heteroaryl group, or a combination thereof.

3. The compound for an organic optoelectronic device as claimed in claim 1, wherein R³ to R⁵ in Chemical Formulae 1 and 2 are each independently hydrogen, a C1 to C30 alkyl group, a C6 to C30 aryl group, or a combination thereof.

4. The compound for an organic optoelectronic device as claimed in claim 1, wherein the substituent represented by Chemical Formula 1 is represented by the following Chemical Formula 1a: 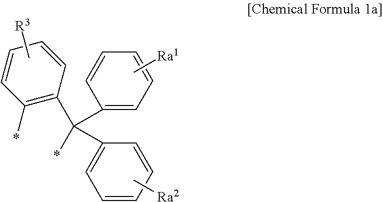
wherein in Chemical Formula 1a:
Ra¹ and Ra² are each independently hydrogen or a C1 to C10 alkyl group, and
R³ is hydrogen, a carbazolyl group, a C1 to C30 alkyl group, a C6 to C30 aryl group, a C2 to C30 heteroaryl group, a C6 to C30 arylamine group, or a combination thereof.

5. The compound for an organic optoelectronic device as claimed in claim 1, wherein the substituent represented by Chemical Formula 2 is represented by one of the following Chemical Formulae 2a to 2c: 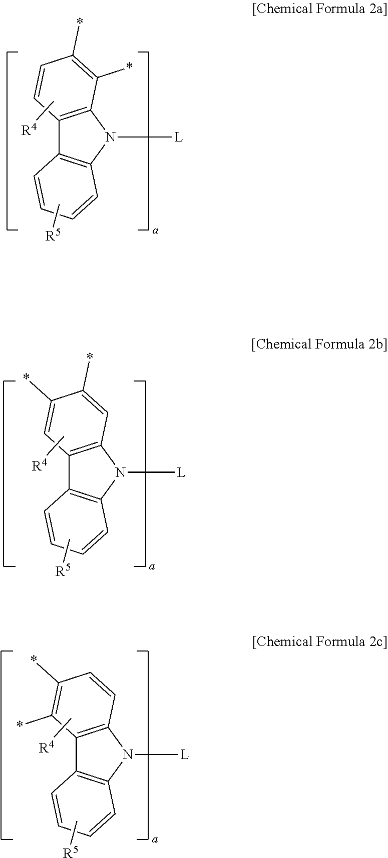
wherein, in Chemical Formulae 2a to 2c:
L is a divalent to heptavalent linking group, the divalent to heptavalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof,
R⁴ and R⁵ are each independently hydrogen, a carbazolyl group, a C1 to C30 alkyl group, a C6 to C30 aryl group, a C2 to C30 heteroaryl group, a C6 to C30 arylamine group, or a combination thereof, and
a is an integer of 2 to 5.

6. The compound for an organic optoelectronic device as claimed in claim 1, wherein the divalent to heptavalent linking group of L of Chemical Formula 2 is derived from a compound represented by one of the following Chemical Formulae 2d to 2j, or a combination thereof: 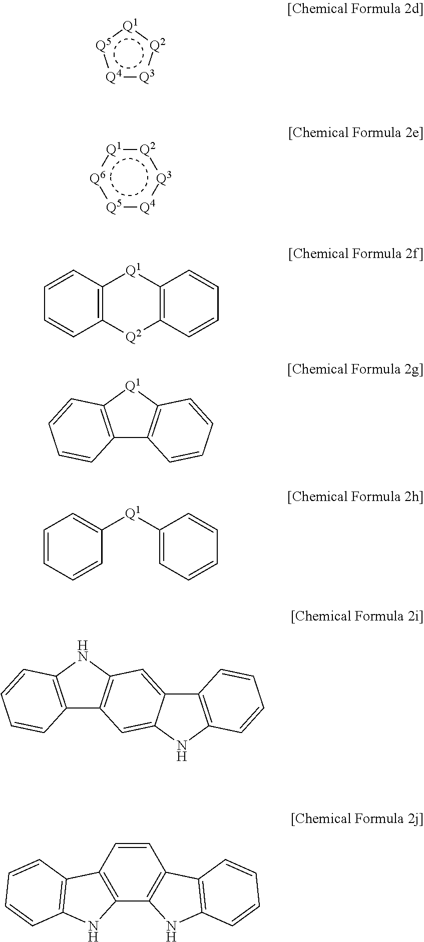
wherein, in Chemical Formulae 2d to 2j:
Q¹ to Q⁶ are each independently a substituted or unsubstituted N atom, a substituted or unsubstituted P atom, a substituted or unsubstituted S atom, a substituted or unsubstituted O atom, a substituted or unsubstituted C atom, or a combination thereof, in which substituted refers to one substituted with an oxide group, a cyano group, a halogen group, a C1 to C30 alkyl group, a C6 to C30 aryl group, a C2 to C30 heteroaryl group, or a combination thereof.

7. The compound for an organic optoelectronic device as claimed in claim 1, wherein, in Chemical Formula 2, a is 2 or 3.

8. The compound for an organic optoelectronic device as claimed in claim 1, wherein the divalent to heptavalent linking group of L of the above Chemical Formula 2 is represented by one of the following Formulae L-1 to L-117:

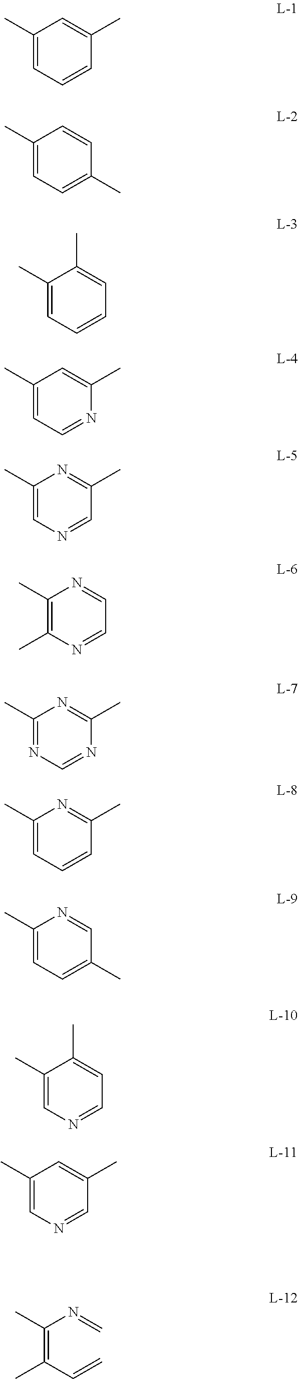

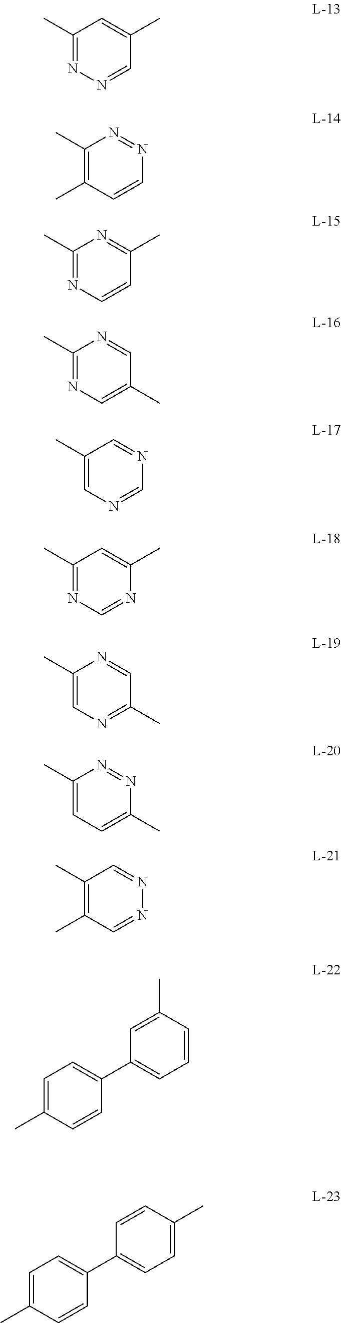

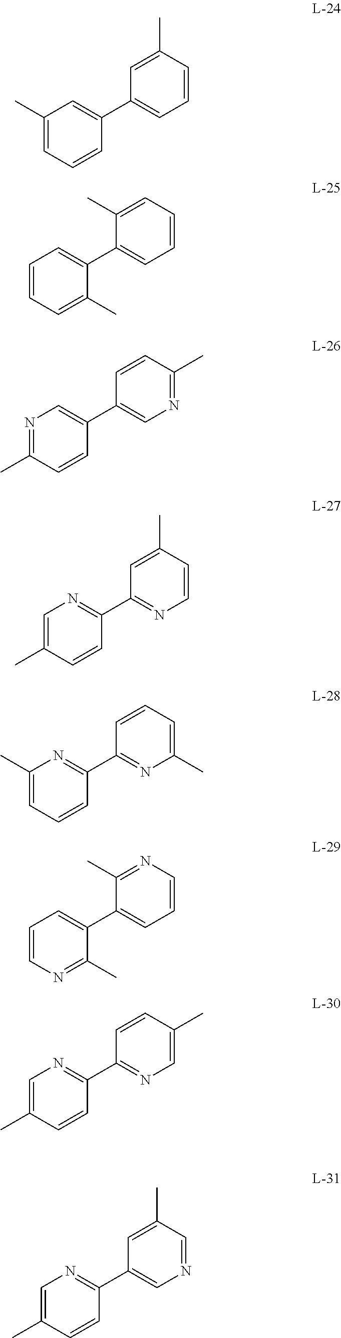

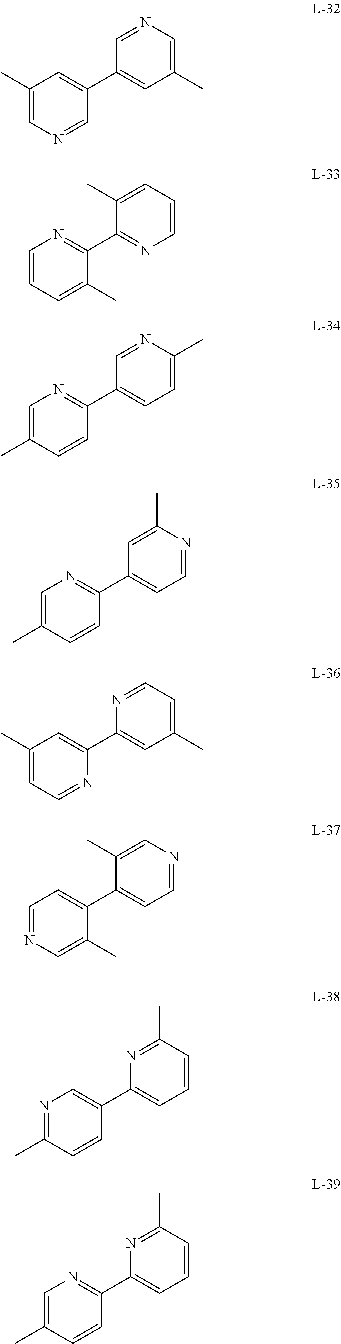

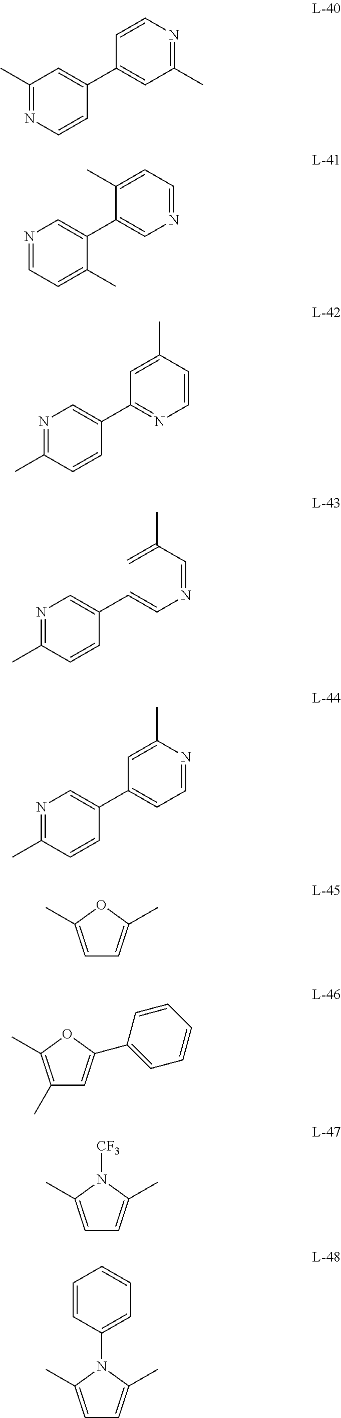

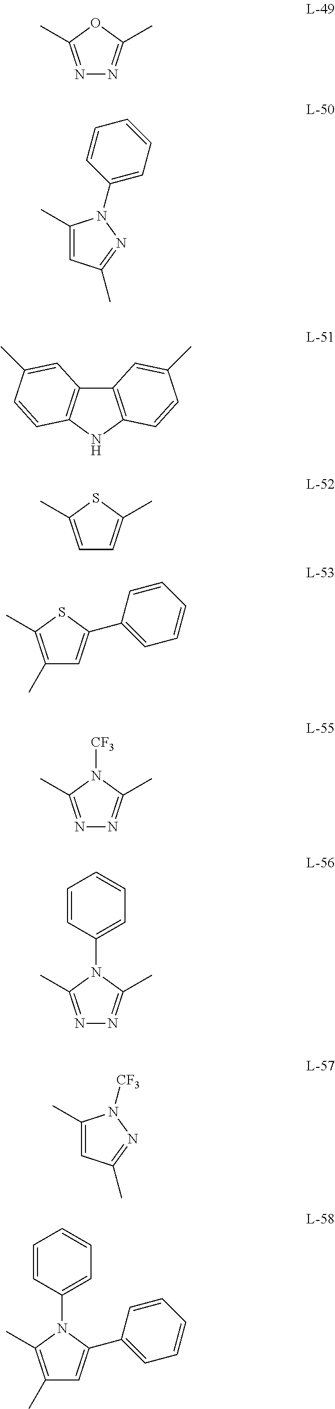

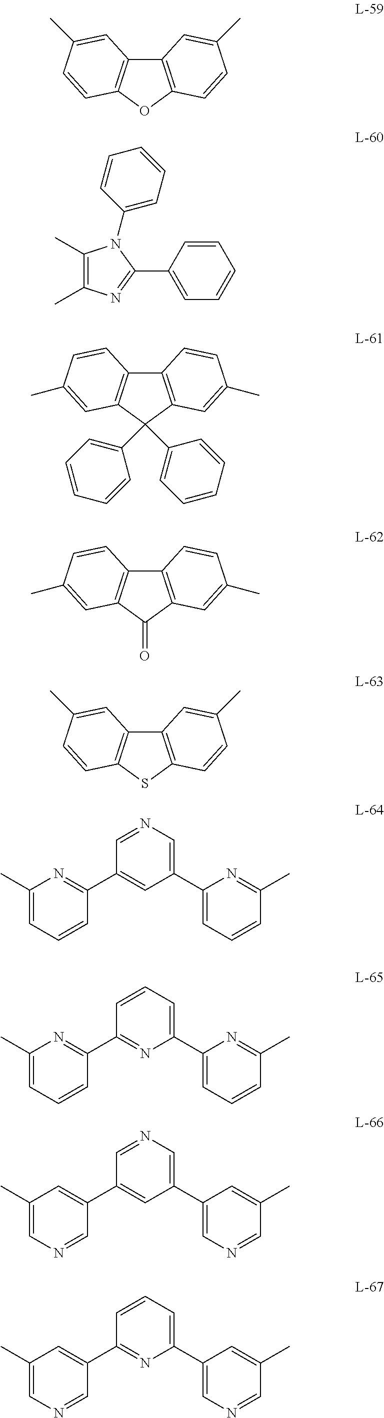

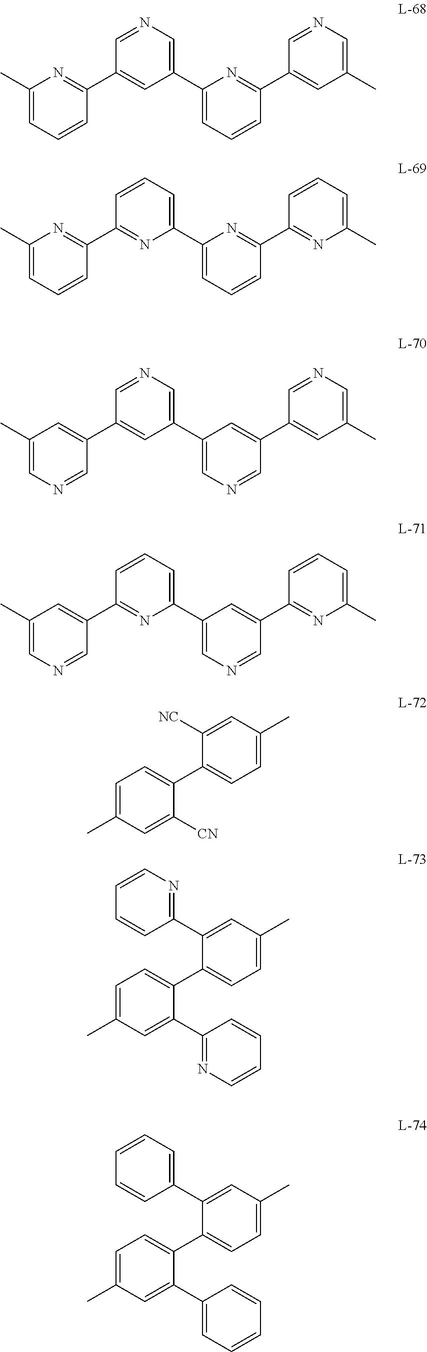

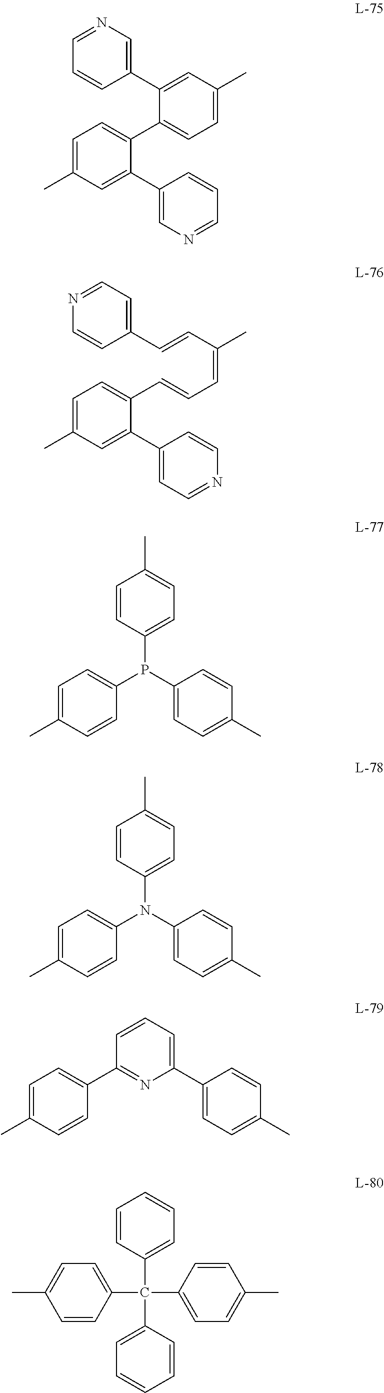

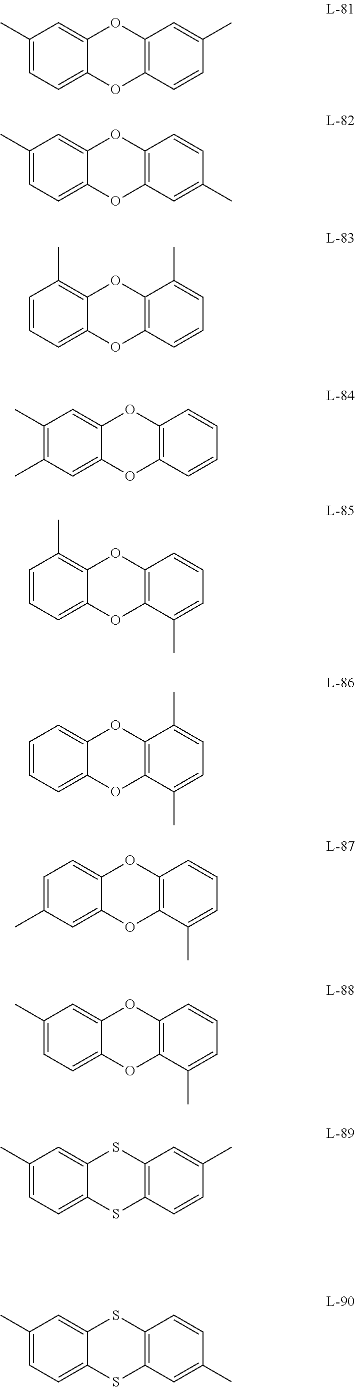

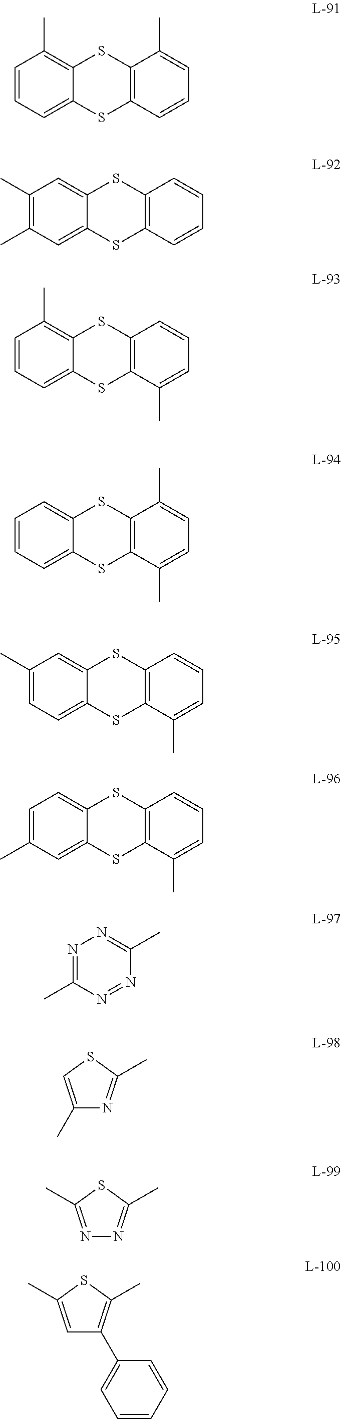

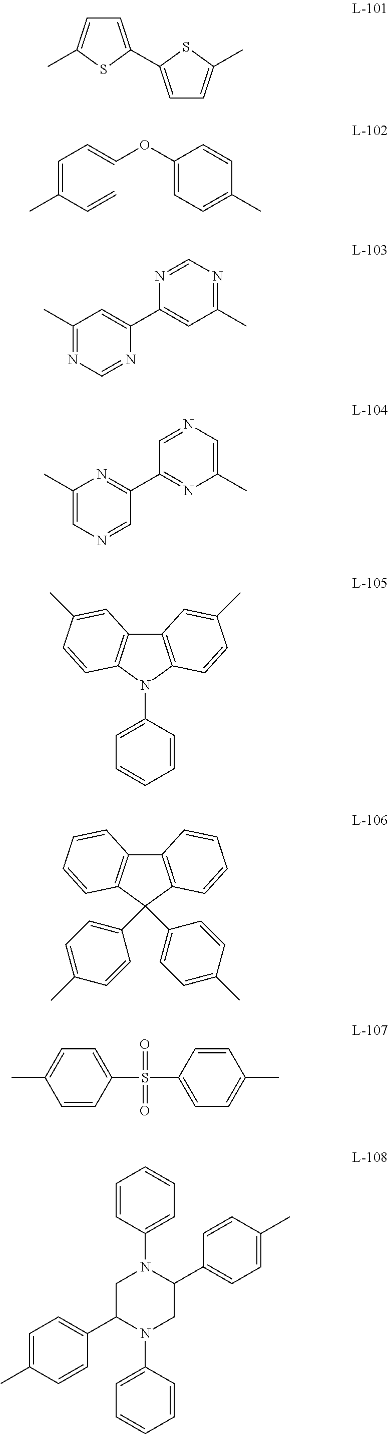

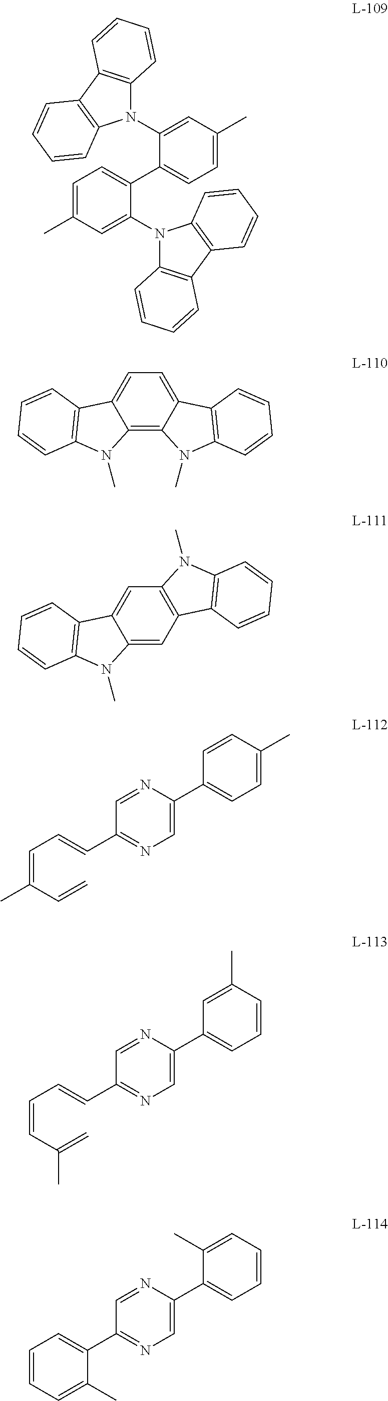

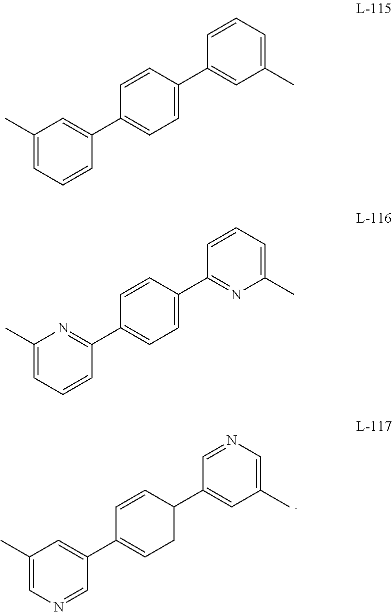

9. The compound for an organic optoelectronic device as claimed in claim 1, wherein the compound for an organic optoelectronic device including the substituents represented by Chemical Formulae 1 and 2 is represented by one of the following Chemical Formulae 3 to 8: 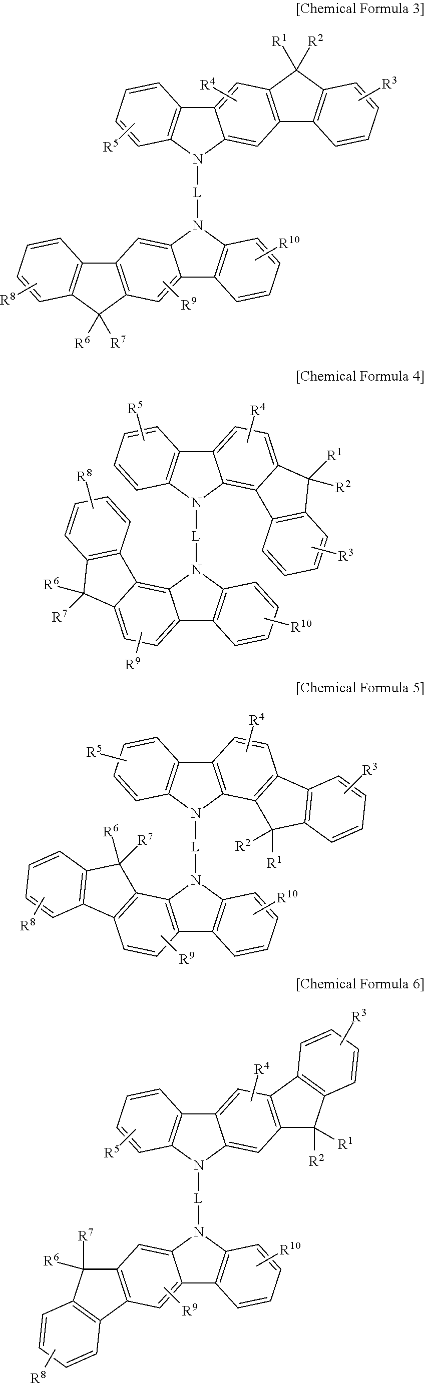 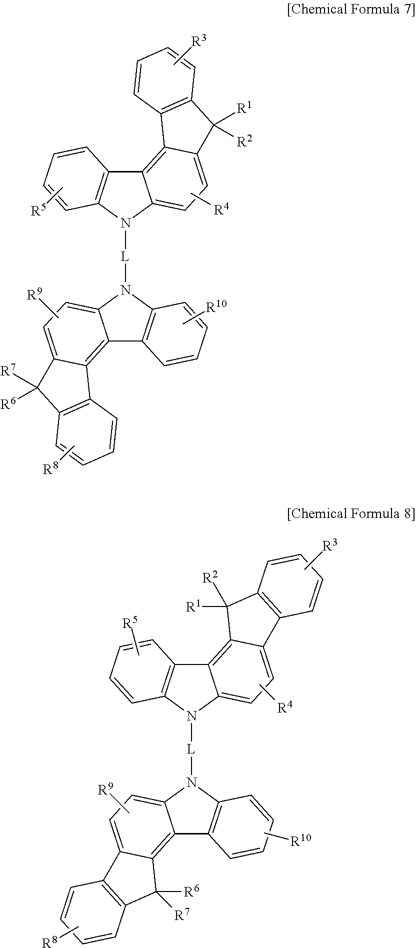
wherein, in Chemical Formulae 3 to 8:
L is a divalent linking group, the divalent linking group including an oxide group, an amino group, a phosphonyl group, a phosphonate group, a sulfonyl group, a sulfonate group, a substituted or unsubstituted C1 to C30 alkylene group, a substituted or unsubstituted C1 to C30 heteroalkylene group, a substituted or unsubstituted C3 to C30 cycloalkylene group, a substituted or unsubstituted C1 to C30 heterocycloalkylene group, a substituted or unsubstituted C6 to C30 arylene group, a substituted or unsubstituted C2 to C30 heteroarylene group, or a combination thereof, and
R¹ to R¹⁰ are each independently hydrogen, a carbazolyl group, a C1 to C30 alkyl group, a C6 to C30 aryl group, a C2 to C30 heteroaryl group, a C6 to C30 arylamine group, or a combination thereof.

10. The compound for an organic optoelectronic device as claimed in claim 1, wherein the compound for an organic optoelectronic device including the substituents represented by Chemical Formulae 1 and 2 is represented by one of the following Chemical Formulae 9 to 33:

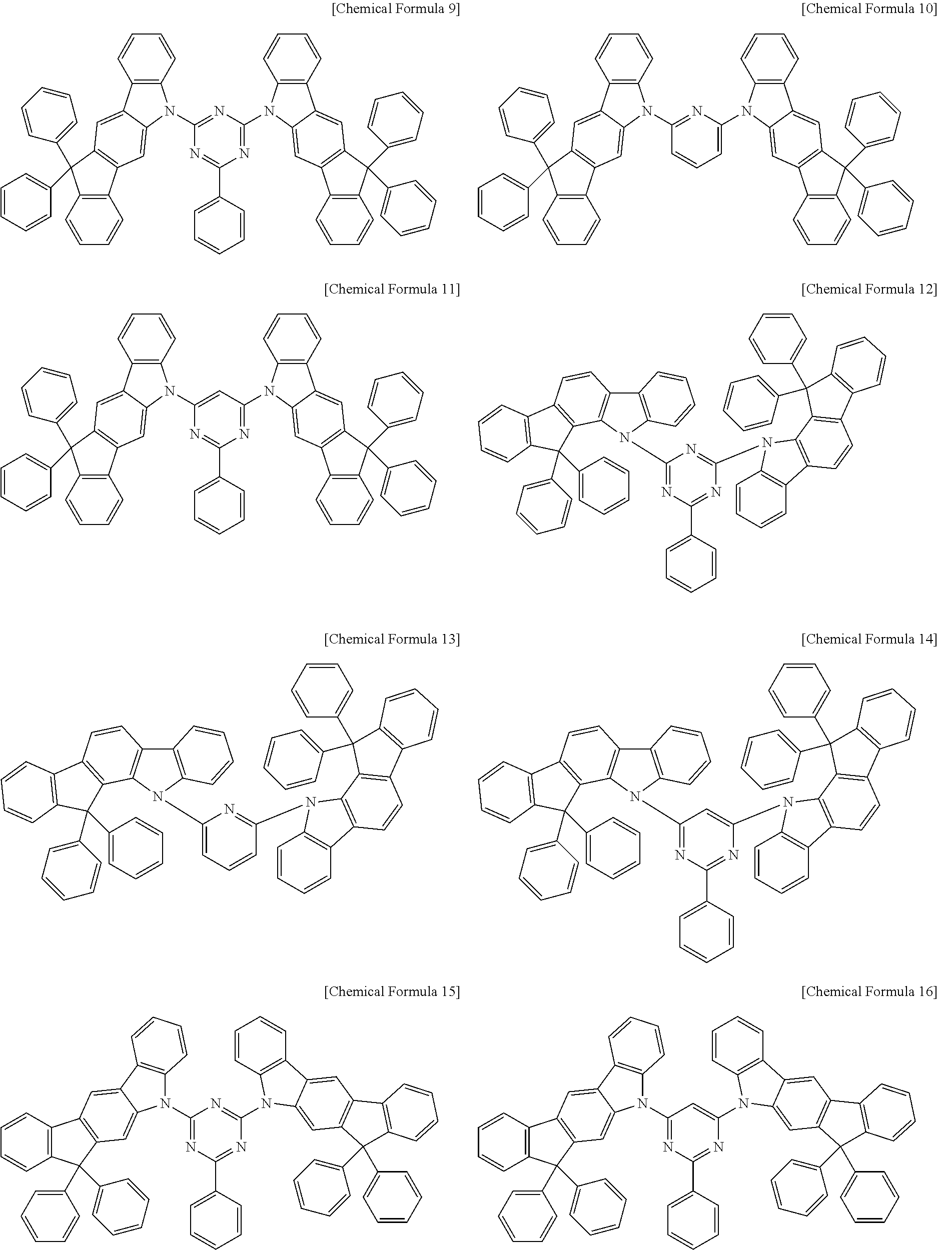

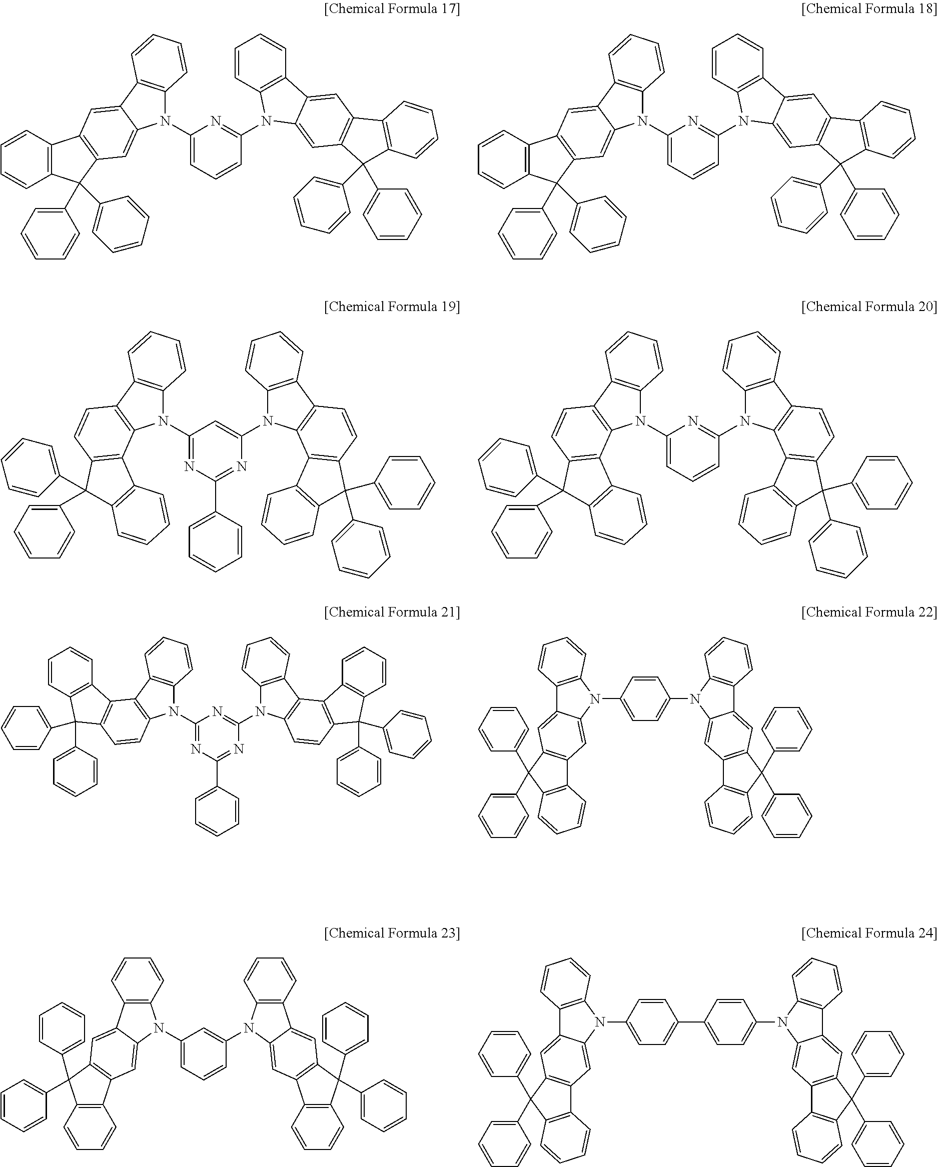

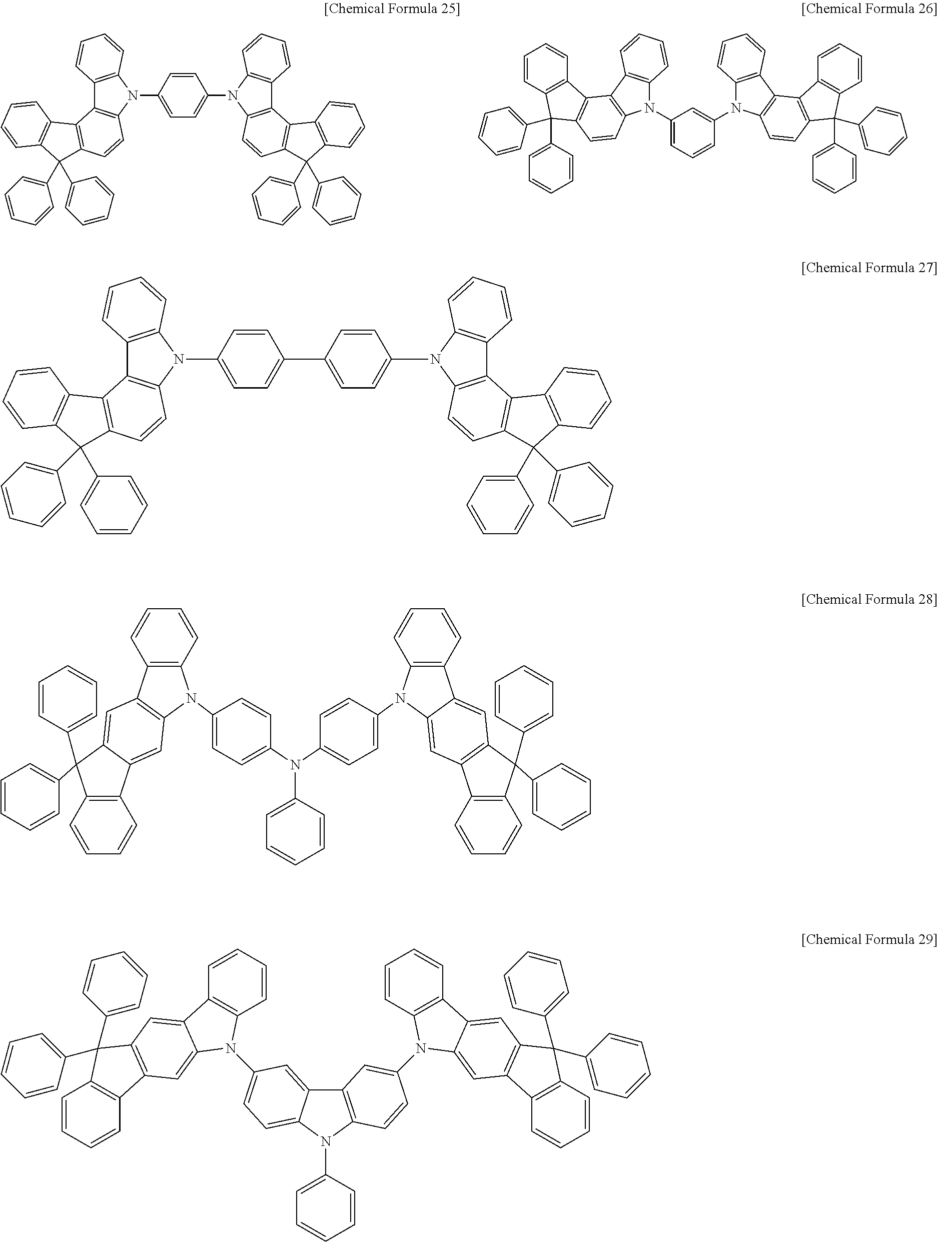

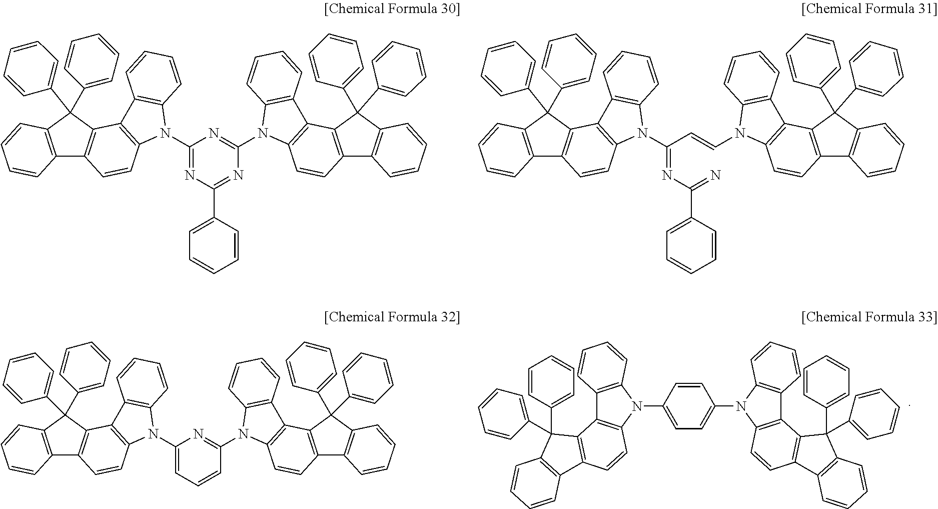

11. The compound for an organic optoelectronic device as claimed in claim 1, wherein the compound for an organic optoelectronic device is a charge transport material or a host material.

12. The compound for an organic optoelectronic device as claimed in claim 1, wherein the compound for an organic optoelectronic device has a thermal decomposition temperature (Td) of about 350 to about 600° C.

13. An organic light emitting diode, comprising:
an anode,
a cathode, and
at least one organic thin layer interposed between the anode and cathode,
wherein the at least one organic thin layer includes the compound for an organic optoelectronic device as claimed in claim 1.

14. The organic light emitting diode as claimed in claim 13, wherein the at least one organic thin layer includes an emission layer, a hole blocking layer, an electron transport layer (ETL), an electron injection layer (EIL), a hole injection layer (HIL), a hole transport layer (HTL), an electron blocking layer, or a combination thereof.

15. A display device comprising the organic light emitting diode as claimed in claim 13.
